(19) United States
(12) Patent Application Publication
(10) Pub. No.: US 2019/0175104 A1
(43) Pub. Date: Jun. 13, 2019

(54) IN-SITU SALIVARY COMPONENT COLLECTION, CONCENTRATION, ISOLATION, ANALYTICS, AND COMMUNICATION SYSTEM

(71) Applicant: Bela Malik, Corpus Christi, TX (US)

(72) Inventor: Bela Malik, Corpus Christi, TX (US)

(21) Appl. No.: 15/836,901

(22) Filed: Dec. 10, 2017

(51) Int. Cl. A61B 5/00; A61B 5/145; A61B 10/00

(52) U.S. Cl. CPC A61B 5/682; A61B 5/076; A61B 10/0051; A61B 5/145

(57) ABSTRACT

A system for in-situ saliva collection and concentration including devices and components configured to be inserted or imbedded in an oral cavity or integrated body sensor of an animal or human. The apparatus includes a receptacle for saliva collection, concentration, isolation, or one or more sensors which are further configured to interface a plurality of one or more customizable functions and applications. A part or entire device can also be configured to be inserted into a “reader,” which is external to the oral cavity. The reader is either “smart” or connected to a “smart device, and is configured to contain one or more sensors. The system includes oral fluid concentration through biochemical and biophysics technologies, and other platforms, analytics and diagnostics to accurately determine health and performance. The invention provides innovative salivary information systems, methods, diagnostic, and performance platforms involving the afore-described devices from information available from oral biomarkers.

### CROSS-REFERENCE TO RELATED APPLICATIONS

This application claims priority to U.S. Pat. App. No. 62/432,658 entitled “In-situ Salivary Component Collection, Concentration, Analytics, and Communication System,” which was filed on Dec. 11, 2016, and U.S. App. No. 62/434,716 entitled “In-situ Salivary Component Concentration, Analytics, Diagnosis, and Athletic Performance System,” which was filed on Dec. 15, 2016. This application is continuation-in-part of U.S. patent application Ser. No. 14/850,713 entitled “Oral Sensor Alerting and Communication System and Developers' Tool Kit,” which was filed on Sep. 10, 2015. We claim priority of this application because this is a continuation-in-part of the patent application Ser. No. 14/850,713 with the emphasis on Concentration Alert which is detailed herein.

### STATEMENT REGARDING FEDERALLY SPONSORED RESEARCH OR DEVELOPMENT

Not applicable.

The present disclosure relates to smart, implantable, insertable, fittable, or attachable oral saliva collection receptacles for in-situ oral fluid component collection, concentration, isolation, detection, analysis, and the integration of such with mobile communications. This system displays mobile communications related alerts, information, and related technologies for both animals and humans referred to herein as In-situ Salivary Component Collection, Concentration, Isolation, Storage, and Communication System (ISCCCISCS).

### BACKGROUND

The background is incorporated herein by reference from the U.S. patent application Ser. No. 14/850,713 entitled “Oral Sensor Alerting and Communication System and Developers' Tool Kit,” which was filed on Sep. 10, 2015, and paragraph numbers 004-006.

### SUMMARY OF THE INVENTION

The present invention provides smart oral cavity devices, systems, and methods relating thereto, as well as auxiliary devices and methods that greatly improve the well-being of animals and humans through innovations in such technologies. This invention provides smart systems, implantable, insertable, fitted, or attachable devices and methods for greatly improving the well-being of animals and humans through innovations in in-situ oral fluid and salivary component collection, concentration, isolation, storage, detection, and biosensor technology. The invention combines it's enhanced, implantable, insertable, fittable, or attachable salivary fluid component collection, concentration, isolation, and storage devices with biosensors and with communications, software management, data management, instant and long-term animal and human analyses, multimedia inputs, visualizations, geometric motion, tracking, kinematics, alerting, therapeutic, electronic medical records and other beneficial systems not previously available.

The In-situ Salivary Component Collection, Concentration, Isolation, Storage, and Communication System (ISCCCISCS) device of the invention includes a receptor for in-situ salivary component collection, concentration, isolation, and storage, which is implantable, insertable, fittable, or attachable securely within a human or an animal's oral cavity. Besides, in-situ salivary component, all oral components could be collected, concentrated, isolated, and stored. The oral components include salivary components, biologics, biologically relevant, drugs, ingested materials, oral blood, and oral microbial. The biologic molecule in this application is any molecule naturally found in or on human or animal body. A biologically relevant molecule is any introduced or non-naturally molecule that has relevance to human or animal bodies.

The ISCCCISC device is configured such that, at least, one or more module of the said device could be removed for further processing, detection, and analysis by a medical device referred here as “reader,” and the system referred herein as In-situ Saliva Component Collection, Concentration, Isolation, and Storage Reader System (ISCCCISRS). The reader herein could be “smart” or could be connected to a smart device. In certain situations, the smart reader could be a cellular phone with accessories. Besides, being smart, the reader could also “read” biological information using sensors and/or accessory functions. In some instances, the light source on the cellular phone could be converted to excitation energy source; whereas, the camera could be converted to a light measuring device such as optical photo, density, or colorimeter, video, photodiode, LED. The camera could be used alone or in combination with other sensors to measure or record changes in biological activity. Furthermore, the touch sensors or pressure sensors in the cellular phone could be used to measure changes in volume, pressure, etc. Accessories could be attached to the cellular phone that contains sensors. The sensors could be selected from the group consisting of, but not limited to, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

The reader could be used when the device cannot be miniaturized to fit in the oral cavity or when saliva component, before detection, needs processing by substances that cannot be used in the oral cavity. The reader or part of the reader could be connected, attached, inserted, or fitted to the ISCCCISCS device either while the device is in the oral cavity or after the device is fully or partially removed from the oral cavity.

It is understood by anyone familiar with arts that ISCCCISCS and ISCCCIRS could be used interchangeably for all embodiments except for automatic or real-time alerts but not near-time alerts.

Both the oral device and reader could contain same or similar sensors and biosensors as described herein e.g., the sensors could be selected from the group consisting of, but not limited to, temperature, blood, pressure, teeth pressure, ionic conductivity, airflow, optimum breathing, oral air-flow, images, optical density, alterations to the oral cavity, surrounding muscle tone, muscle weakness, heart rate, heart rhythms, respiration rate, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

The ISCCCISCS component of the invention provides for communication systems and alerting technology that links a multitude of saliva-based biological information inputs together. This method of gathering biological information from implantable, insertable, fittable, or attachable oral devices provides the basis for a real-time or near-time snapshot of an animal or human's health and well-being.

Accordingly, ISCCCISCS and an oral biosensor alert and communication system and other methods and devices related to and used in conjunction in addition to that are provided which address the needs and provide the advantages outlined herein.

Salivary fluids are not available for real-time, near time, or at-point-of-care for study and evaluation of systemic disorders, and there is a lack of an effective means for real-time, near-time, or at point-of-care diagnosis. One major reason for this is very dilute salivary components, which make detection difficult especially for real-time, near time, or at-point-of-care analysis. The invention presented here would resolve this significant problem.

Salivary fluids are not available for real-time, near time, or at-point-of-care for study and evaluation of systemic disorders, and there is a lack of an effective means for real-time, near-time, at point-of-care diagnosis. One major reason for this is the interference between salivary components and also between food, plaque, shredded mucosal cells, oral bleeding, and other oral debris and salivary components. The invention presented here would resolve this significant problem by separating and isolating salivary components from each other and food, plaque, mucosal cells, oral blood, and other oral components.

The ISCCCISCS device of the invention includes a smart in-situ salivary collection and concentration component and a biosensor receptacle for one or more oral biosensors which is implantable, fittable, or attached securely within a human or an animal's oral cavity.

In a further embodiment, the invention provides an ISCCCISCS and a reader where the “reader” contains one or more sensors and could also contain at least one interface with a network capable of utilizing the information obtained from the collected and concentrated salivary component and its interaction with at least one oral biosensor.

Also provided is an ISCCCISCS or ISCCCIRS device according to the invention where this oral fluid collection, concentration, detection, and sensor devices are connected to “smart” medical devices in response to alerts and/or signals from the ISCCCISCS or ISCCCIRS.

The ISCCCISCS oral device or ISCCCIRS could contain biosensors and sensors that would read or collect the bio-information within saliva or in some embodiments the concentrated and isolated salivary components would be sent to a laboratory for further analysis. Whether the biological information gathered would be collected by sensors within the oral cavity, by the reader, or send to a laboratory for further analysis, would depend upon one or more factors related to salivary component and sensors. These factors are a component concentration in both the saliva and blood, sensitivity of the sensor, component's biochemical and biophysical properties, requirement for further processing, user need, ease of manufacturing, production, and regulations. Based on sensor sensitivity and other factors that would be determined during development and manufacturing, the concentrate and isolated component would be sent to a laboratory for further processing and detection.

In a further embodiment, the invention provides an ISCCCCISC system. The system includes the above-described receptacle, at least one collection, concentration device, storage, detection, and/or oral biosensor within the receptacle. The device could also be smart and contain at least one interface with a network capable of utilizing the information obtained from the collected and concentrated salivary component interaction with at least one oral biosensor.

In a further embodiment, the invention provides an ISCCCISCS or ISCCCIRS system wherein the system includes the above-described smart receptacle that contains at least one permeable membrane, semi-permeable membrane, micro-pump, paper microfluidics, milli-fluidics module, microfluidics module, or nanofluidics chip or module.

In a further embodiment, the invention provides an ISCCCISC system wherein the system includes the above-described receptacle that contains at least a buffer, preservative, or biologic stabilizing compounds.

In a further embodiment, the invention provides an ISCCCISCS wherein the system includes the above-described smart receptacle and a saliva calibration chip or modules to measure the amount of saliva removed and normalize data gathered.

In a further embodiment, the invention provides an ISCCCISCS system wherein the system includes the above-described smart receptacle and a saliva storage chamber or module.

In a further embodiment, the invention provides an ISCCCISC system wherein the system includes the above-described receptacle and a salivary component reader for near-time detection and analysis, and the reader is outside of the oral cavity.

In an aspect of the invention, a device is provided which includes the said receptacle as described before. The receptacle is configured to be inserted into an oral cavity of an animal or human. The receptacle is configured to serve one or more functions within the animal or human's oral cavity wherein the one or more functions could be customized by physicians, veterinarians, patients, animal owners, users, caretakers, and others. The customizable functions can utilize mechatronics and can be integrated with sensors selected to measure and diagnose one or more medical health biometrics. ISCCCISCS imbedded oral cavity detection system could be utilized in conjunction with including, but not limited to, the one or more functions the said receptacle is configured to serve, e.g., is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information. These systems and sensors are used so that they cannot be swallowed.

The device includes one or more ISCCCISCS and/or sensors contained within or upon the receptacle or multiple receptacles.

In a further embodiment, the invention provides an ISCCCISCS including the above-described receptacle could be attached and de-attached to, but not limited to, the one or more functions the said receptacle is configured to serve, e.g., is selected from the group consisting of consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information. These systems and sensors are used so that they cannot be swallowed.

In a further embodiment, the invention provides an ISCCCISCS wherein the system includes the above-described receptacle and dental veneers and recreational appearance. The device could serve a cosmetic function by improving the appearance of a smile through increasing teeth appearance or appearance of recreational function.

In another embodiment of the invention, the ISCCCISCS can streamline and integrate performance measurements such as, but not limited to, various geometric models, visualization, complex spatial-temporal relations, human and animal facial and physical relationships (individually and group), data associations (i.e., pixels, auditory, motion, optimum breathing, oral air-flow, accelerometers, gyroscope, metabolic biosensors, high-definition video capture, body-wearable sensors, RFIDs, readers, positioning, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple, various degrees of dynamic alerting, tracking, positioning, multi-media, analytics, historical and other comparative data inputs, communications and platforms). Collectively, these inputs can be synced and integrated with all forms of data capture. The ISCCCISCS can provide important real-time or near time analytics to correct or modify a player's motion, behavior for individuals, team sports or organizational groups for animals and humans.

In a further embodiment, the invention provides an ISCCCISC system including the above-described smart receptacle, one or more oral fluid collection and/or storage chambers contained within, attached, or upon the receptacle and at least one interface with a network configured to utilize the information obtained from the one or more sensors.

In a further embodiment, the invention provides an ISCCCISC system including the above-described smart receptacle, one or more concentration modules or chips contained within, attached, or upon the receptacle and at least one interface with a network configured to utilize the information obtained from the one or more sensors.

In a further embodiment, the invention provides an ISCCCISCS including the above-described smart receptacle, one or more sensors contained within, attached, or upon the receptacle and at least one interface with a network configured to utilize the information obtained from the one or more sensors.

It is understood by anyone familiar with the art that the dental device could be customized or semi-customizable.

It is understood by anyone familiar with the art that the ISCCCISCS or ISCCCIRS or parts thereof could be attached or de-attached from the dental device.

It is understood by anyone familiar with the art that independent of wireless storage; the data could be stored in any ISCCCISCS or ISCCCIRS device through any digital storage device, connector, or mechanism.

In yet a further embodiment of the invention, a method is provided for obtaining sensor data from a human and/or an animal. The method includes the steps of inserting an oral ISCCCISCS receptacle configured to serve one or more functions within a human or an animal's oral cavity. The receptacle contains or receives within or upon it one or more ISCCCISCS receptacle capable of providing information relevant to the health or a physiological characteristic of the human or animal. The method further involves activating or monitoring one or more ISCCCISCS and sensors to obtain or to analyze the information relevant to the health or a physiological characteristic of the human or animal, and transmitting, at least, some portion of the health or physiological information or analysis to a network capable of utilizing the information obtained. The one or more functions the said receptacle is configured to serve, e.g., is selected from the group consisting of consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, oral medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information. These extraction systems and sensors are used so that they cannot be swallowed and used in any combination thereof.

In an embodiment, this system could be used to Insitu Blood Component Collection, Concentration, Isolation, and Storage Communication System (IBCCCISCS) or Insitu Blood Component Collection, Concentration, Isolation, and Storage Reader System (IBCCCISRS). In this embodiment, IBCCCISCS or IBCCCISRS could be used in combination with several transdermal extractions techniques are used for transdermal extractions including, but not limited to, electrically, thermal, vibrational or ultrasound, mechanically, chemical enhancers, and electrochemical gradient assisted devices. Examples of electrically assisted are electroporation, ionophoresis, and reverse iontophoresis of thermally assisted are a laser, radio frequency heating, chemical heating, and mechanical heating. Ultrasound-assisted are sonophoresis and phonophoresis; mechanical assisted are micro-needles and tape-stripping; chemical and electrochemical gradient based are salt, natural, human-made polymers and compound assisted diffusion, surfactants, enzymes, salts, chemical- or enzyme-peels, nanoparticles, polymer-chain, microparticulate, etc. It is understood by anyone familiar with arts that oral blood could substitute saliva for all embodiments and situations described herein.

The recognition component in these systems and methods of the invention, often called a cellular receptor, can use, e.g., biomolecules from organisms or receptors modeled after biological systems to interact with an analyte of interest. This interaction can be measured by a bio-transducer which outputs a measurable signal proportional to the presence of a target analyte in the sample, which in this case is oral saliva.

### BRIEF DESCRIPTION OF THE DRAWINGS

The presently disclosed subject matter will be better understood from reading the following description of non-limiting embodiments, with reference to the attached drawings, wherein below:

FIG. 1A is a depiction which exemplifies an ISCCCISCS smartwatch, necklace, smart horse-bit, smart horse lip or cheek clip-on, smart pacifier, equipped with secure wireless communication capability and retrieves information in the women's oral cavity according to the invention.

FIG. 1B is a depiction which exemplifies an ISCCCISCS smart pacifier, horse-bit, lip or cheek clip-on equipped with secure wireless communication capability and retrieves information through ISCCCISCS device in the baby's, dog's, or horse's oral cavity according to the invention.

FIG. 2 exemplifies the structure of a sensor or biosensor as may be used in embodiments of the invention.

FIG. 3. Unfixed dental devices are defined as ones not permanently attached to the jaw bone, but as possibly attached to the gum or teeth.

FIG. 4. Fixed or permanent dental devices: In certain embodiments, these dental devices are attached to the jaw bone through metal, dental enamel, etc.

FIG. 5 depicts location of various salivary glands and their ducts.

FIG. 6A depicts embodiments which exemplify the locations of ISCCCISCS on a retainer.

FIG. 6B depicts embodiments which exemplify the locations of ISCCCISCS on a mouth guard.

FIG. 6C depicts embodiments which exemplify the locations of ISCCCISCS on a horse-bit.

FIG. 6D depicts embodiments which exemplify the locations of ISCCCISCS on a horse lip or cheek clip-on.

FIG. 6E depicts embodiments which exemplify the locations of ISCCCISCS on a pacifier.

FIG. 6F depicts embodiments which exemplify the locations of ISCCCISCS on medical equipment.

FIG. 7 depicts embodiments of ISCCCISCS which exemplify neutral and charged molecular movement across a semi-permeable membrane, which is utilized in the present invention.

FIG. 8 depicts embodiments which exemplify collection, concentration, storage, and detection of the salivary fluids.

FIG. 9 depicts embodiments which exemplify ISCCCISCS dynamic alerting software and secure networks, in accordance with embodiments of the present invention.

### DETAILED DESCRIPTION OF THE INVENTION

The preceding summary, as well as the following detailed description of certain embodiments, will be better understood when read in conjunction with the appended drawings. As used herein, an element or step recited in the singular and proceeded with the word “a” or “an” should be understood as not excluding the plural of said elements or steps, unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

As used herein the terms “insertable”, “implantable”, “attachable”, “fittable”, “embeddable”, “temporarily insertable” “permanently insertable”, “temporarily implantable”, “permanently implantable”, “permanently attachable”, “temporarily attachable”, “temporarily fitable”, “permanently fitable”, “temporarily embeddable” and “permanently embeddable” refer to means of securely inserting and attaching in or to, or fastening a device, such as being adhered to, cemented, affixed or otherwise securely attached to a surface or object.

As used herein, the term “receptacle” refers to a device or container that receives, retains, has within, or holds something.

As used herein, the term “smart” means a device or object that performs one or more functions of a computer or information system, such as data storage, calculation, Internet access and information transmission.

Described herein the broader respects of the SMART INSITU SALIVARY COMPONENT COLLECTION, CONCENTRATION, ISOLATION, STORAGE, AND COMMUNICATION SYSTEM (ISCCCISCS) of the invention, which includes a device configured to be inserted and securely attached or fitted in an oral cavity of an animal or human. The device includes a receptacle for a saliva component collection, concentration, isolation, storage modules, cartilages, or chips, and a sensor. The device could be smart. The receptacle is preferably configured to serve one or more functions within the animal or human's oral cavity without being swallowed. The device could also include one or more sensors contained within or upon the receptacle, and also at least one interface with a network configured to utilize the information obtained from the one or more sensors or from one or more platforms providing additional information or capabilities networked with the system.

The ISCCCISCS device of the invention includes a receptor for in-situ salivary component collection, concentration, isolation, and storage module or chip which is implantable or attached or fittable securely within a human or an animal's oral cavity. At least one or more module of the said device could be removed for further processing, detection, and analysis by a smart medical device referred here as “reader,” and the system referred herein as INSITU SALIVARY COMPONENT COLLECTION, CONCENTRATION, ISOLATION, STORAGE READER SYSTEM (ISCCCISRS).

In ISCCCISCS device of the invention, in addition to in-situ salivary component, all oral components could be collected, concentrated, isolated, and stored. The oral components include salivary components, biologics, biologically relevant, drugs, ingested materials, oral blood, and oral microbial. The biologic molecule in this application is any molecule naturally found in or on human or animal body. A biologically relevant molecule is any introduced or non-naturally molecule that has relevance to human or animal bodies.

Described herein in broader respects is the ISCCCISRS of the invention includes a device configured to be inserted and securely attached in an oral cavity of an animal or human. The device includes a smart receptacle for a saliva collection, concentration, and storage modules, cartilage, or chips. The receptacle is preferably configured to serve one or more functions within the animal or human's oral cavity without being swallowed. The system also includes one or more sensors contained within or upon an external detector or “reader,” and also at least one interface with a network configured to utilize the information obtained from the one or more sensors or from one or more platforms providing additional information or capabilities networked with the system.

The ISCCCISCS device of the invention includes a receptor for in-situ salivary component collection, concentration, isolation, and storage module or chip which is implantable, insertable, fittable, or attachable and de-attachable securely within a human or an animal's oral cavity. At least one or more module of the said device could be removed for further processing, detection, and analysis by a medical device referred here as “reader,” and the system referred herein as Insitu Saliva Component Collection, Concentration, Isolation, and Storage Reader System (ISCCCISRS). The reader herein could be “smart” or could be connected to a smart device. In certain situations, the smart reader could be a cellular phone with accessories. Besides, being smart, the reader could also “read” biological information using sensors and/or accessory functions. In some instances, the light source on the cellular phone could be converted to excitation energy source; whereas, the camera could be converted to a light measuring device such as optical photo, density, or colorimeter, video, photodiode, LED. In some situations, the camera could be used alone or in combination with other sensors to measure or record changes in biological activity, the pressure sensors in the cellular phone could be used to measure changes in volume, pressure, etc. Accessories could be attached to the cellular phone that contains sensors. The sensors could be selected from the group consisting of, but not limited to, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

The reader could be used when the device cannot be miniaturized to fit in the oral cavity or when saliva component, before detection, needs processing by substances that cannot be used in the oral cavity. The reader or part of the reader could be connected, attached, inserted, or fitted to the ISCCCISCS device either while the device is in the oral cavity or after the device is fully or partially removed from the oral cavity.

It is understood by anyone familiar with arts that ISCCCISCS and ISCCCIRS could be used interchangeably for all embodiments except for automatic real-time alerts but not for near-time alerts. Both the oral device and reader could contain same or similar sensors and biosensors as described herein e.g., the sensors could be selected from the group consisting of, but not limited to, temperature, blood, pressure, teeth pressure, ionic conductivity, airflow, optimum breathing, oral air-flow, images, optical density, alterations to the oral cavity, surrounding muscle tone, muscle weakness, heart rate, heart rhythms, respiration rate, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

Furthermore, in some situations, the collected, concentrated, and isolated samples could be sent to a laboratory for more processing and detection of the biologics. Situations determinations would be done based on user, development, manufacturing, and regulations requirements.

Saliva components are not available for accurate real-time and at-point-of-care analysis study and evaluation of several systemic disorders, and there is a lack of an effective means for real-time diagnosis. One major reason for this is very low levels of salivary components in saliva, and therefore, many salivary components are present at sub-detectable levels, which make detection difficult especially for real-time analysis. Moreover, interference and hindrance from plaque, oral bleeding, other salivary components, mucosal cells, food, and other oral debris, etc. could cause difficulties with accurate detection and analysis of salivary components. Therefore, salivary components separation and isolation from each other and plaque, mucosal cells, food, and other oral debris, oral bleeding, etc. could enhance accurate detection. The invention presented here would resolve these significant problems. Therefore, the purpose here is to in-situ separate, isolate, and concentrate salivary components from each other such that the desired components become concentrated and detectable

The ISCCCISCS device of the invention includes a smart in-situ salivary component collection and concentration and biosensor receptacle for one or more oral biosensors which are implantable, fittable, or attached securely within a human or an animal's oral cavity.

In a further embodiment, the invention provides an ISCCCISCS and a reader where the “reader” contains one or more sensors and could also contain at least one interface with a network capable of utilizing the information obtained from the collected and concentrated salivary component interaction with at least one oral biosensor.

Also provided is an ISCCCISCS or ISCCCIRS device according to the invention where this oral fluid collection, concentration, detection, and sensor devices are connected to “smart” medical devices in response to alerts and/or signals from the ISCCCISCS or ISCCCIRS.

In a further embodiment, the invention provides an ISCCCISCS or ISCCCIRS device could measure, calibrate, or normalize collected or concentrated salivary components.

In a further embodiment, the invention provides an ISCCCISCS or ISCCCIRS system. The system includes the above-described smart receptacle, at least one collection, concentration device, storage, detection, or oral biosensor contained within the receptacle. The device could also contain at least one interface with a network capable of utilizing the information obtained from the collected and concentrated salivary component interaction with at least one oral biosensor.

The ISCCCISCS oral device or ISCCCIRS could contain biosensors and sensors that would read or collect the bio-information within saliva or in some embodiments the concentrated and isolated salivary components would be sent to a laboratory for further analysis. Whether the information gathered would be collected by sensors within the oral cavity, by the reader, or send to a laboratory for further analysis, would depend upon a variety of factors. These factors could be the component concentration in the saliva and/or blood, sensitivity of the sensor, component's biochemical and biophysical properties, requirement for further processing, user need, ease of manufacturing, production, regulations, etc. Based on sensor sensitivity and other factors that would be determined during development and manufacturing, the concentrate and isolated component would be sent to a laboratory for further processing and detection. Still, components with higher concentration and sensor sensitivity would be contained within the device; whereas, components present at sub-detection levels in the salivary concentrate would be sent to a laboratory for further analysis, and further processing and detection, for accurate detections. Based on sensor sensitive and other factors that would be determined during development and manufacturing would be “reader” by a provided reader.

In a further embodiment, the invention provides an ISCCCCISC system. The system includes the above-described receptacle, at least one collection, concentration device, storage, detection, and/or oral biosensor within the receptacle. The device could also be smart and contain at least one interface with a network capable of utilizing the information obtained from the collected and concentrated salivary component interaction with at least one oral biosensor.

In a further embodiment, the invention provides an ISCCCISCS or ISCCCIRS system wherein the system includes the above-described smart receptacle, and contains at least one or more of the following: permeable membrane, semi-permeable membrane, non-permeable, synthetic membranes, biological membranes, coated membranes, uncoated membranes, color changing membranes, microfiltration membrane, ultra-filtration membranes, nano-filtration membranes, osmotic membranes, reverse osmotic membranes, ionic or charged membranes, neutral membranes, size-exclusion membranes, affinity membranes, adsorption membranes, extracting membranes, binding membranes, hydrophilic membranes, hydrophobic membranes, electrostatic attraction and repulsing membranes, polar membranes, non-polar membranes, lipophilic membranes, lipophobic membranes, coated membranes, resistant membranes, capillary action membranes, suction generating membranes, micro-pumps, absorbent materials, pressure generating membranes, surface tension generating membranes, energy releasing membranes, energy absorbing membranes, and reactive membranes. It is understood by anyone familiar with the arts that the membranes can be replaced by coated micro- or nano-beads, non-coated micro-, nano-beads or gel.

In a further embodiment, the invention provides an ISCCCISCS system wherein the system includes food sensors, edible coating, edible membranes, edible nano-beads, and other materials used in food and agriculture industries. These safe materials could be ingestion without hazardous.

It is understood by anyone familiar with arts that one or more sensors could be coupled with ISCCCISCS or ISCCCIRS to gather health and performance information.

It is understood by anyone familiar with arts that food debris, plaque, mucosal cells and cell debris could hinder the interaction between sensors and biologically relevant molecule. This hindrance could be removed by the clearing of the debris in the salivary fluids by the membranes mentioned above and beads such that the interactions could be more accurate.

In a further embodiment, the invention provides an ISCCCISCS or ISCCCIRS system wherein the system includes the milli-fluidics module or chip or paper, paper microfluidics module or chip, microfluidics module or chip or paper, or nanofluidics chip or module or paper or micro-pump.

It is understood by anyone familiar with the arts that salivary component/s biochemical and biophysical properties would determine choice and arrangements of various membranes, micro-beads, gel, milli-fluidics, microfluidics, nanofluidics module or chip or paper, and/or micro-pump to separate, isolate, concentrate, and collect oral fluid.

In a further embodiment, the invention provides an ISCCCISCS or ISCCCIRS system wherein the system includes the above-described smart receptacle contains at least contain a buffer, preservative, and/or biologic stabilizing compounds. These buffers, preservatives, and biologics, biologic stabilizing compounds could be in liquid, solid, or gaseous forms.

In a further embodiment, the invention provides an ISCCCISCS wherein the system includes the above-described smart receptacle and a saliva calibration chip or modules to measure the amount of saliva removed and normalize data gathered.

In a further embodiment, the invention provides an ISCCCISCS system wherein the system includes the above-described smart receptacle and a saliva storage chamber, cartridge, or module.

In a further embodiment, the invention provides an ISCCCIRS system wherein the system includes the above-described smart receptacle and a salivary component reader for real-time and at point-of-care detection and analysis and the reader is outside of the oral cavity.

It is understood by anyone familiar with the art that independent of wireless storage; the data could be stored in any ISCCCISCS or ISCCCIRS device through any digital storage device, connector, or mechanism.

The device includes one or more ISCCCISCS and/or sensors contained within or upon the receptacle or multiple receptacles.

In an aspect of the invention, a device is provided which includes the said receptacle as described before. The receptacle is configured to be inserted into an oral cavity of an animal or human. The receptacle is configured to serve one or more functions within the animal or human's oral cavity wherein the one or more functions could be customized by physicians, veterinarians, patients, animal owners, users, caretakers, and others. The customizable functions can utilize mechatronics and can be integrated with sensors selected to measure and diagnose one or more medical health biometrics. ISCCCISCS imbedded oral cavity detection system could be utilized in conjunction with including, but not limited to, the one or more functions the said receptacle is configured to serve, e.g., is selected from the group consisting of consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information. These systems and sensors are used so that they cannot be swallowed and any combination thereof.

In yet a further embodiment of the invention, a method is provided for obtaining sensor data from a human and/or an animal. The method includes the steps of inserting an oral ISCCCISCS receptacle configured to serve one or more functions within a human or an animal's oral cavity. The receptacle contains or receives within or upon it one or more ISCCCISCS receptacle capable of providing information relevant to the health or a physiological characteristic of the human or animal. The method further involves activating or monitoring the one or more ISCCCISCS and sensors to obtain or analyze the gathered information relevant to the health or a physiological characteristic of the human or animal, and transmitting at least some portion of the health or physiological information or analysis to a network capable of utilizing the information obtained. The one or more functions the said receptacle is configured to serve, e.g., is selected from the group consisting of consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information. These extraction systems and sensors are used so that they cannot be swallowed.

In an embodiment, this system could be used to Insitu Blood Component Collection, Concentration, Isolation, and Storage Communication System (IBCCCISCS) or Insitu Blood Component Collection, Concentration, Isolation, and Storage Reader System (IBCCCISRS). In this embodiment, IBCCCISCS or IBCCCISRS could be used in combination with several transdermal extractions techniques are used for transdermal extractions including, but not limited to, electrically, thermal, vibrational or ultrasound, mechanically, chemical enhancers, and electrochemical gradient assisted devices. Examples of electrically assisted are electroporation, ionophoresis, and reverse iontophoresis of thermally assisted are a laser, radio frequency heating, chemical heating, and mechanical heating. Ultrasound-assisted are sonophoresis and phonophoresis; mechanical assisted are micro-needles and tape-stripping; chemical and electrochemical gradient based are salt, natural, human-made polymers and compound assisted diffusion, surfactants, enzymes, salts, chemical- or enzyme-peels, nanoparticles, polymer-chain, microparticulate, etc. It is understood by anyone familiar with arts that oral blood could substitute saliva for all embodiments and situations described herein.

In a further embodiment, the invention provides an ISCCCISCS and ISCCCIRS system, including the above-described smart receptacle that could be attached and de-attached. One or more functions of the said receptacle is configured to serve, but not limited to, e.g., is selected from the group consisting of consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for sports and recreational performance, animal sports and recreational performance, and other medical diagnostics, and analytics function and other functions and applications in order to provide accurate physiological information. These systems and sensors are used so that they cannot be swallowed.

In a further embodiment, the invention provides an ISCCCISCS system wherein the system includes the above-described smart receptacle and dental veneers and recreational appearance. The device could serve a cosmetic function by improving the appearance of a smile through increasing teeth appearance such as color or covering chipped or broken teeth and/or protecting weak and sensitive teeth. The cosmetic appearance of the recreational function is wherein the teeth can have the appearance of distorted and discolored teeth for example during Halloween and such recreational activities.

In a further embodiment, the invention provides an ISCCCISCS system including the above-described smart receptacle, one or more oral fluid collection and/or storage chambers contained within, attached, or upon the receptacle and at least one interface with a network configured to utilize the information obtained from the one or more sensors.

In a further embodiment, the invention provides an ISCCCISCS system including the above-described smart receptacle, one or more concentration modules or chips contained within, attached, or upon the receptacle and at least one interface with a network configured to utilize the information obtained from the one or more sensors.

In a further embodiment, the invention provides an ISCCCISCS including the above-described smart receptacle, one or more sensors contained within, attached, or upon the receptacle and at least one interface with a network configured to utilize the information obtained from the one or more sensors.

It is understood by anyone familiar with the art that the dental device could be customized or semi-customizable.

It is understood by anyone familiar with the art that the ISCCCISCS or ISCCCIRS could be attached or de-attached from the dental device.

In certain embodiments, the system is set up wherein, at least, one of the one or more functions the said receptacle is configured to serve, e.g., is selected from the group consisting of consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information. These systems and sensors are used so that they cannot be swallowed and providing a recreational or sports function.

Additionally, the system may further include one or more sensors contained within or upon one or more receptacles located within or upon the animal or human's body networked with the oral cavity device.

The network units of the system include ones capable of utilizing the information obtained from the one or more sensors and having functions including, but not limited to, data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, analyzing historical or previous data from same or different individual or team, visualizing and/or presenting development platforms.

Sensors may include sensors for a biologic, a biologically relevant molecule, blood pressure, blood component, pH, respiration associated gases, pulse, gyroscopic measurement, accelerometer measurement, and the network capable of utilizing sensor information is configured to have the information transduced, amplified, or processed and a signal from the network transmitted through a RFID tag to an RFID reader on an accessory attached.

The system/s includes one or more network units which can be configured to carry out a functionality including or consisting of signaling bi-directional transmissions to a secure server through one or more of WiFi, Bluetooth, GPS, MiFi, Internet of things, and NFC, temporarily storing information in the smart device, and bi-directionally transmitting alerts to pre-selected devices or pre-selected personnel. Further network units employable in the system include or consist of, one or more RFID components, one or more cloud applications, a real-time or near-time slumber to alert mode, a manual control diagnosis mode, a programmed automated diagnosis mode, a geographic analysis mode, a disease-specific or situational alerting mode, a function by which the one or more oral sensors is activated and inactivated by another sensor, device or remote controller, and transmission through WiFi or other wireless mode.

In additional embodiments, the system includes at least one auxiliary smart sensor receptacle not configured to be inserted in an oral cavity of an animal or human. The system can include a network configured to analyze one or more performance parameters of a team sport or group activity.

The system including the auxiliary smart sensor receptacle can also include a network including one or more RFID components, micro- and nano-electronics, micro- and nano-enabled energy harvesting, micro- and nano-energy storage, micro- and nano-devices, micro- and nano-electronics, micro- and nano-enabled energy harvesting, micro- and nano-energy storage, micro- and nano-devices, micro- and nano-timer, micro- and nano-devices, micro- and nano-programmable processors, micro- and nano-memory, micro- and nano-integrated power management, micro- and nano-programmable hardware, micro- and nano-wireless communication capabilities across multiple frequencies located in a mouth or integrated outside of a mouth, one or more cloud applications, a real-time or near-time slumber to alert mode, a manual control diagnosis mode a programmed automated diagnosis mode, a geographic analysis mode, a disease specific or situational alerting mode.

The system can include one or more medical devices or medication dispensers. Further enhancements include a fully integrated treatment facility, a system in which one or more sensors is activated by another sensor, device or remote controller, network modes including transmission through WiFi or other wireless modes, and systems in which at least one auxiliary smart sensor receptacle for a sensor is configured to serve one or more secondary functions within the animal or human's oral cavity.

The one or more functions served by the receptacle of the device can include or consist of functions selected from the group consisting of, but not limited to, replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information and providing a recreational or sports function, health analytics, diagnostic analytics, performance analytics; integration of body sensors, health-devices, nano-particles, and sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement and greater precision and analytics.

The device receptacle is insertable, e.g., by micro- and nano-clips, frames, brackets, sealants, dental composites, bonds, adhesives, adhesive strips, cement, suction or vacuum for adherence, wires, bands, glues, embedment, injection, printing, tattooing, or any combination thereof.

In these devices, the receptacle or a part of the receptacle can be configured to be removable, self-removable, self-installable, attachable, de-attachable, coverable with an air-tight material, and configured so that the one or more sensors are integral to the receptacle.

The sensors in the provided devices can include, e.g., sensors of blood pressure, levels of a predetermined biologic, chemical or medication or their metabolites, salivary components, or oral fluid components, or respired gases.

The sensors in the provided devices can include sensors which measure a salivary, blood, lymph node, bone, or tooth constituent, sensors which measure a predetermined biologic, sensors which measure a predetermined biologic including or consisting of DNA, RNA, telomeres, methylated or otherwise modified DNA or RNA, proteins, immunoglobins, antibodies, histones, peptides, modified proteins, neuro-peptides, pigments, blood chemicals, pH, and enzymes, sensors which measure dissolved gases, including oxygen, carbon dioxide, carbon monoxide, ammonia, sulphur, or an alcohol-containing gas, sensors which measure a lipids profile, sensors which measure a chemical molecule, sensors which measure a salt, an alcohol, a metabolite, an anion, a cation, water, a sugar, a protein, or a lectin, sensors which measure a drug or a medication, sensors which measure cells, the one or more sensors measures cells, cancerous cells, biomarkers for an oral or systemic infectious disease, biomarkers for drug abuse, biomarkers for a metabolic disease, biomarkers for malnutrition, biomarkers for obesity, biomarkers for a cardiovascular disease, biomarkers for atherosclerotic, biomarkers for infection, biomarkers for auto-immune and other immune diseases, biomarkers for stroke, biomarkers for AIDs, biomarkers for multiple sclerosis, biomarkers for periodontal diseases, biomarkers for brain-function disorders, dementia, memory loss, depression, mental disease, Alzheimer's disease, mentally-challenged disorders, nervous system disorders, tracking or wandering, and other psychology and neurological disorders, biomarkers for bleeding, head and neck injuries, biomarkers for Sjogen's syndrome, biomarkers for oxidative stress, biomarkers for allergies, biomarkers for cancer, biomarkers for skeletal and muscle diseases, biomarkers for genetic diseases, biomarkers for renal diseases, biomarkers for osteoporosis, biomarkers for fatigue, biomarkers for stress, biomarkers for sleep deprivation or sleep apnea, biomarkers for fertility, pregnancy, ovulation, and reproductive system disorders, biomarkers for cystic fibrosis, biomarkers for respiratory or pulmonary diseases, biomarkers for diabetes and ketoacidosis, biomarkers for inflammation, biomarkers for age-related diseases, biomarkers for dehydration, biomarkers for halitosis, biomarkers for alcohol consumption, alcoholism or drug consumption or drug addiction, biomarkers for hypoxia, smoking-related diseases, toxins, or pollutants, biomarkers for poor-gait, biomarkers for Crohn's disease, biomarkers for Cushing disease, biomarkers of stress, biomarkers for dental caries, biomarkers for blood and circulatory disorders, biomarkers for ear, nose, and throat diseases, biomarkers for taste, Ageusia, Hypogeusia, or Dysgeusia, biomarkers for bad-breath related diseases biomarkers for chewing or mastication, biomarkers for digestive disorders, biomarkers for hepatic diseases, spleen, gall-bladder and pancreatic diseases, biomarkers for urinary system disorders, biomarkers for integumentary system diseases, biomarkers for endocrine, lymphatic, and excretory diseases, sensors which measure a cell surface component or a cellular marker or component, sensors which measure a pathogen or a microbe, sensors which measure administered foreign materials, medications, diagnostic molecules, drugs, biologically sensitive, derived, bio-mimics, or bioengineered molecules, sensors which measure an ingested molecule or its metabolite, including wherein ingested molecule is a pathogen, a microbial, an ingested toxin, an ingested allergen, an ingested food constituent, including a nutrient, a micronutrient, a fat molecule, a carbohydrate molecule, a sugar molecule, a protein molecule, or an amino acid, sensors which measure ingested medications, ingested foreign material, ingested drugs, an ingested diagnostic molecule, an ingested biologically sensitive molecule, an ingested nanoparticle, an ingested derived molecule, a bio-mimic, or an ingested bioengineered molecule, sensors which interact with at least one disease-related biomarker, such as, e.g., a disease-related biomarker related to a disease diagnosable by a sensor which includes or consists of sensors of blood pressure, core body temperature, heart rate, levels of a predetermined biologic, chemical or medication or their metabolites.

The device, as described herein, can be securely attached, fitted, inserted within the oral cavity of an animal or human by a number of means, including one or more of being fixedly inserted, imbedded, fitted, fixed, implanted, fastened, joined, associated, coupled, linked, banded, united, mounted, combined, glued, adhered, cemented, or suction or vacuum for adherence, or firmly connected by mouth, e.g., lips, teeth, etc., or hands or parts or accessories thereto of either.

In a particular embodiment, the device can include an interface with at least one sensor or nano-particles not located within the oral cavity. The systems and devices described as part of the ISCCCISCS as laid out in this application can be applied to obtain extensive sensor data and analysis more accurately from sub-optimal concentrations of salivary components without interference from other oral components providing much-needed information and assistance, as detailed herein. In particular, a method for obtaining such data from an animal (pets) or human is provided. The method includes the steps of locating or inserting a device configured to be inserted or fitted and securely attached in an oral cavity of an animal or human. The device includes a receptacle for ISCCCISCS or ISCCCISRS, the receptacle being configured to serve one or more functions within the animal or human's oral cavity without being swallowed. In the method, the said receptacle is inserted already containing, or, alternatively, receives one or more ISCCCISCS or ISCCCISRS with one or more sensors after insertion, capable of providing or receiving information or analysis relevant to the animal or human. The method includes activating and/or monitoring the one or more sensors, and transmitting or receiving at least some portion of the information or analysis to, from or among a network or networks capable of utilizing the information or analysis.

In the above-described method, the one or more functions of the receptacle can include or consist is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information or providing a function related to health analytics, diagnostic analytics, performance analytics; integration of body sensors, health-devices, nano-particles, sports and performance sensors on inanimate objects and sports equipment; customizable developers' tool kit for biosensors, sensors, performance, medical analytics, oral and systemic body diagnosis; integrated, pre-integrated and post-integrated, platforms; any type of medium, secure bidirectional media, multiple media, video, audio, 3D, printing, reporting, analytics, reporting, metadata diagnosis, or providing functions with geometric tracking, communication networks, analytics, alerting, kinematics for individuals, team sports, organizational groups, animals and humans, communications, software management, data management, instant and long term animal and human analyses, multimedia inputs, visualizations, geometric motion, tracking, kinematics, alerting, therapeutic, electronic medical records, historical analysis, time stamped data, reporting and feedback, positioning, the integrated video can be synced with all wearables and other biosensors in order to produce computer-generated precise movement or greater precision and analytics.

In a particular aspect of the above method, at least one of the one or more functions of the receptacle includes or is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth and any combination thereof; wherein at least one of the functions includes providing an aligning, fixing of malpositioned teeth or jaws, or other corrective function and the receptacle is retainers, braces, space maintainers, headgear, palatal expanders, fixed prostheses, braces, dental wires, partial retainers or full retainers; wherein at least one of the functions includes providing a cosmetic or cleansing function and the receptacle is veneers, whitening and cleansing strips, and professional, amateur, or lay-person cleansing tools and equipment, and whitening or cleansing traces; wherein at least one of the functions includes providing a pacifying function for infants, children, adults, elderly, and animals, and the receptacle is a infant, children, adult, and animal pacifier, pacifier-like device; wherein at least one of the functions includes providing sleep-aid and preventing snoring and obstructive sleep apnea function, mandibular advancement devices (MAD), tongue retaining devices (TRD), continuous positive airway pressure (CPAP) combination CPAP/dental sleep device therapy; physiological, natural and/or grinding protective function and the receptacle is a night guard or partial guard; wherein at least one of the functions is providing a recreational, athletic performance, or sports function and the receptacle is a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, assisting in proper breathing, eating or swallowing, providing a tongue thrust dental guard function, tongue protector, an attachment to a tooth, inner-lips, or inner-cheeks, inner lip clip, inner cheek clip, an insert in a gum, gum-cover, a smart gauge, smart dipstick, smart rod, smart stick; horse-rider navigation and communication function, horse-bit; vital sign measuring device, a thermometer; diagnostic and therapeutic function, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, oral surgical, medical devices, and hospitalization equipment function, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, providing a cosmetic and cleansing function, assisting in proper breathing, eating or swallowing, providing a tongue thrust dental guard function, chew toy for babies and animals, providing a sports and recreational, such as flexible, inflexible, temporary, or permanent ISCCCISCS and electronic for analytical and other functions and applications in order to provide accurate physiological information and providing a recreational or sports function.

In a particular aspect of the above method, at least one of the one or more parts of ISCCCISCS oral receptacle includes, but not limited to, is selected from the group consisting of membrane, semi-permeable membrane, non-permeable, synthetic membranes, biological membranes, coated membranes, uncoated membranes, color changing membranes, microfiltration membrane, ultra-filtration membranes, nano-filtration membranes, osmotic membranes, reverse osmotic membranes, ionic or charged membranes, neutral membranes, size-exclusion membranes, affinity membranes, adsorption membranes, extracting membranes, binding membranes, hydrophilic membranes, hydrophobic membranes, electrostatic attraction and repulsing membranes, polar membranes, non-polar membranes, lipophilic membranes, lipophobic membranes, coated membranes, resistant membranes, capillary action membranes, the suction generating membranes, micro-pumps, absorbent materials, the pressure generating membranes, surface tension generating membranes, energy releasing membranes, energy absorbing membranes, and reactive membranes. It is understood by anyone familiar with the arts that the membranes can be replaced by coated micro- or nano-beads, non-coated micro-, nano-beads or gel.

In various embodiments of the method, information can be transmitted securely to a plurality of remote devices monitoring the animal or human, or information can be transmitted securely to a plurality of remote devices monitoring a plurality of animals or humans.

In some embodiments of the method, independent to wireless ISCCCISCS and ISCCCISRS can contain storage, the data could be stored in any ISCCCISCS or ISCCCISRS device through any digital storage device, connector, or another medium integral to or separate from the blood or saliva monitoring oral device.

The network capable of utilizing the information obtained from, the one or more, sensors can further consist of one or more network units having the function of data storage, data retrieval, data synthesis, alert programs, data management, characterization, filtering, transformation, sorting, processing, modeling, mining, inspecting, investigation, retrieval, integrating, dissemination, qualitative, quantitative, normalizing, clustering, correlations, computer derived values and ranges, simple or complex mathematical calculations and algorithms, statistical, predictive, integrative, interpretative, exploratory, abnormality seeking, data producing, comparative, historical or previous from same or different individual or team, visualizing or presentation development platforms.

The method can include network units which utilize preset ranges, dynamic preset ranges, or degrees of alerts from preset ranges for medical or performance analysis. Additionally, the described method can include network units which utilize biosensor or sensor measurements for pre-integration and post-integration analyses, as known by those skilled in such arts.

In various embodiments of the method, ISCCCISCS could be replaced by ISCCCISRS. In these embodiments, the reader could be “smart” or could be connected, attached, inserted, or fitted to a smart device such as a cellular phone with accessories.

The method can also involve inserting the oral ISCCCISRS receptacle or a part of the said receptor into the reader too “read or detect” biological information using sensors and/or accessory functions. The receptor could be first processed before inserting into the reader.

In various embodiments of the method, the light source in the cellular phone could be converted to excitation energy source; whereas, the camera could be converted to a light measuring device such as optical photo, density, or colorimeter, video, photodiode, LED. The camera could be used alone or in combination with other sensors to measure or record changes in biological activity, the pressure sensors in the cellular phone could be used to measure changes in volume, pressure, etc. Accessories could be attached to the cellular phone that contains sensors. The sensors could be selected from the group consisting of, but not limited to, contact, non-contact temperature, infrared, pressure of gas or liquid, absolute pressure differential pressure, vacuum pressure, gauge pressure, conductive rubber pressure, lead zirconate titanate pressure, Polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel (a light sensor and an active amplifier), light dependent resistors, optical filters, fluorescence, phosphorescence, sound, resonant, humidity, changes in humidity (measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity), light, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, temperature, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

The method can also involve inserting the receptacle into the gums and measuring blood chemicals using oral or gum blood. The method can include the receptor containing one or more modules for blood extraction using transdermal extractions modules selected from the group consisting of, but not limited to, electrically assisted such as electroporation, ionophoresis, and reverse ionophororesis; thermal assisted such as laser, radio frequency heating, chemical heating, and mechanical heating, vibrational or ultrasound assisted such as sonophoresis and phonophoresis, mechanically assisted such as micro-needles and tape-stripping, chemical enhancers, and electro-chemical gradient assisted devices such as salt, natural, man-made polymers and compound assisted diffusion, surfactants, enzymes, salts, chemical- or enzyme-peels, nanoparticles, polymer-chain, microparticulate, etc.

In certain embodiments of this system, the animal is a pet, including a dog or cat, or other animals. In embodiments of this system involving pets, the auxiliary device can be a smart collar. Moreover, in systems involving pets, in specific embodiments, the system can include a database compilation of one or more attributes of animals having at least one similar characteristic to the pet and which connects via WiFi to an accessible portal and wherein the system provides diagnostic information regarding the pet. Similarly, for pets, the system can include a database compilation of one or more of the pet's biological or physiological attributes.

Generally, the system can include a historical database of the animal or human as to one or more characteristics from which comparisons or analyses are configured to be made, or a database of animals or humans having a common characteristic to the animal or human on which the smart device is located and for which a predetermined comparison is configured to be made.

In another embodiment, the auxiliary device comprises a smart device configured to be associated with one or more team members of a team wherein the auxiliary smart device comprises one or more sensors configured to obtain information from the one or more team members and is configured to transmit the information or analysis derived therefrom directly or indirectly to a network.

The system can be set up for use with an individual to obtain information from the individual and transmit it or analysis derived from it directly or indirectly to a network.

The system network can interface with a mobile device which in turn provides sensor information or analysis to the individual user, who then receives information feedback regarding a physiological characteristic of a current activity he is engaged in, such as running, jogging, walking, sleeping, or a physical characteristic involved with playing a sport.

The system may utilize a network configured to analyze one or more performance parameters of a team sport or group activity. Additionally, in an additional embodiment, the system can include a network configured to provide an electronic medical records functionality.

The system as described above can include full server access, and the system can be configured to analyze individual or team sports performance as it relates to various body components and sensors.

In one option of the system, the system includes one or more of a digital storage device or full connectivity capability configured to analyze individual and team sports performance as it relates to various body component sensors for post-play analysis and review.

### DETAIL DESCRIPTION OF THE DRAWINGS

The presently disclosed subject matter will be better understood from reading the following description of non-limiting embodiments, with reference to the attached drawings, wherein below:

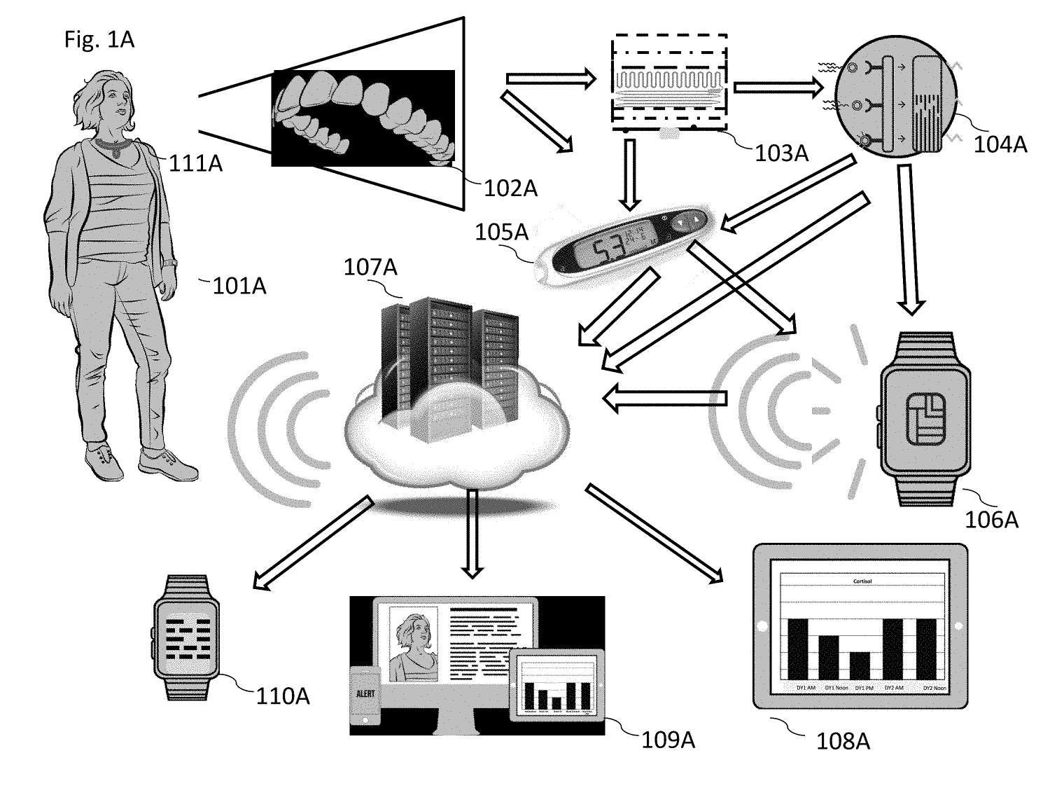

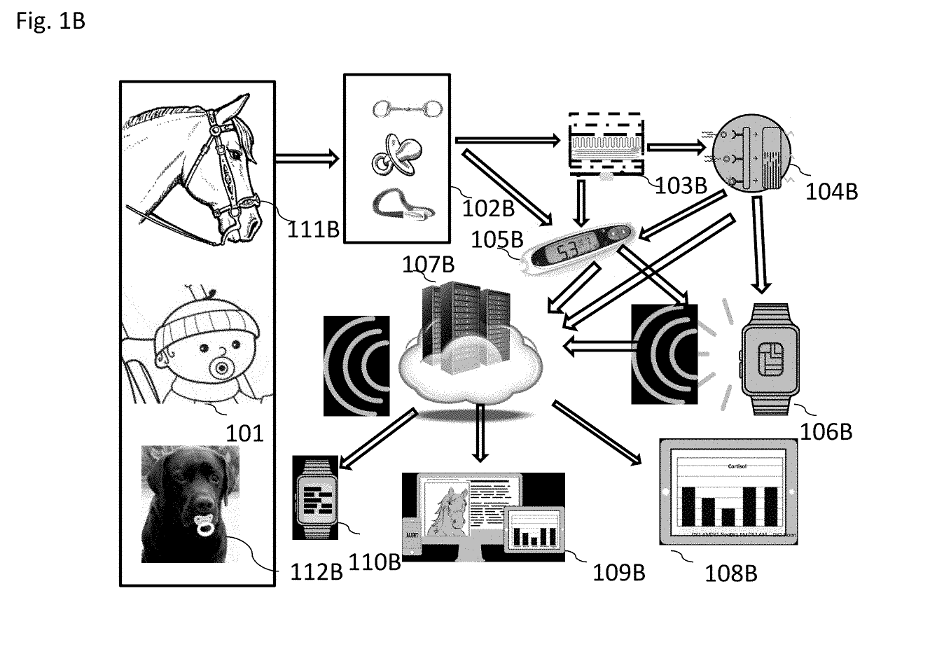

FIG. 1A is a depiction which exemplifies an ISCCCISCS smartwatch or necklace equipped with secure wireless communication (Bluetooth, ZigBee, Wi-Fi, MiFi, Wi-Fi Direct, Internet of Things etc.) capability and retrieves information through ISCCCISCS device in the women's oral cavity according to the invention. In these embodiments of the described ISCCCISCS system, a human, here exemplified by a woman 101A, can have biosensors attached to her teeth through a retainer, or another dental device, 101A, 102A, 111A. Biologics or biologically relevant molecule including, but not limited to, blood sugar or glucose, respiration rate, hydration, or electrolyte would be collected and concentrated and then either detected by a sensor or by reader 103A, 104A, 105A. Alternately, gathered information can be transmitted via the Internet of things where internet communication chips with their IP address and provides the ability to transfer data over a network contained in the ISCCCISCS in oral cavity device or in the “reader.” In some embodiments, information gathered could be stored in ISCCCISCS or in the “reader.” The smartwatch, accessories, or wearables transmits the oral cavity information to one or more remote PC laptops, tablets, smart-phones, wearable, or reader communication devices through the owner's Wi-Fi, MiFi, Wi-Fi Direct, Internet of Things in accordance with embodiments of the present invention. ISCCCISCS device could be inserted in the oral cavity to be bathing in the blood to measure blood glucose levels, blood composition, medication, etc. As needed, the information or signal can then be transduced, amplified, and processed 104A. The resulting signal can be transmitted, e.g., through a RFID tag or blue tooth 5, to a RFID reader or a piconet for blue tooth network on an accessory, smart jewelry, clothing, watch, other accessories, on, in, or around the woman, exemplified here by a smartwatch 106A. The ISCCCISCS system can include an RFID tag reader or blue tooth chip placed within or in proximity to any part of the oral cavity, temporarily or permanently. The signal is then transmitted to a secure server or cloud 107A. Information could be stored or sent from the server or cloud to several different smartphones, smart watches, computers, and others. The alert can be dispatched to any computer-aided device or emergency dispatch if the ISCCCISCS system detects higher than average or abnormal metabolic ranges, for example.

FIG. 1B is a depiction which exemplifies an ISCCCISCS smart bridle or baby necklace equipped with secure wireless communication ((Bluetooth, ZigBee, Wi-Fi, MiFi, Wi-Fi Direct, Internet of Things etc., etc.) capability and retrieves information through ISCCCISCS device in the baby's or horse's oral cavity according to the invention. In these embodiments of the described ISCCC system, a human, here exemplified by a baby 101B, or animals, exemplified here by a dog with pacifier 112B or horse, can have ISCCCISCS and biosensors attached to the pacifier mouth-piece, chew toy, horse-bit, horse cheek or lip clip, or another dental device, 101B, 102B, 111B. Biologics or biologically relevant molecule including, but not limited to, blood sugar or glucose, respiration rate, hydration, or electrolyte would be collected and concentrated and then either detected by a sensor or by reader 103B, 104B, 105B. Alternately, gathered information can be transmitted via the Internet of things where internet communication chips with their own IP address and provides the ability to transfer data over a network contained in the ISCCCISCS in oral cavity device or in the reader. In some embodiments, information gathered could be stored in ISCCCISCS or in the reader. The smartwatch, accessories, or wearables transmits the oral cavity information to one or more remote PC laptops, tablets, smart-phones, wearable, or reader communication devices through the owner's Wi-Fi, MiFi, Wi-Fi Direct, Internet of Things etc. in accordance with embodiments of the present invention. ISCCCISCS device could be inserted in the oral cavity to be bathing in the blood to measure blood glucose levels, blood composition, medication, etc. As needed, the information or signal can then be transduced, amplified, and processed 104B. The resulting signal can be transmitted, e.g., through a RFID tag or blue tooth 5, to a RFID reader or a piconet for blue tooth network on an accessory, smart jewelry, clothing, watch, other accessories, on, in, or around the woman, exemplified here by a smart watch 106B. The ISCCC system can include an RFID tag reader or blue tooth chip placed within or in proximity to any part of the oral cavity, temporarily or permanently. The signal is then transmitted to a secure server or cloud 107B. Information could be stored or sent from the server or cloud to several different smartphones, smartwatches, computers, and others. The alert can be dispatched to any computer-aided device or emergency dispatch if the ISCCCISCS system detects higher than average or abnormal metabolic ranges, for example.

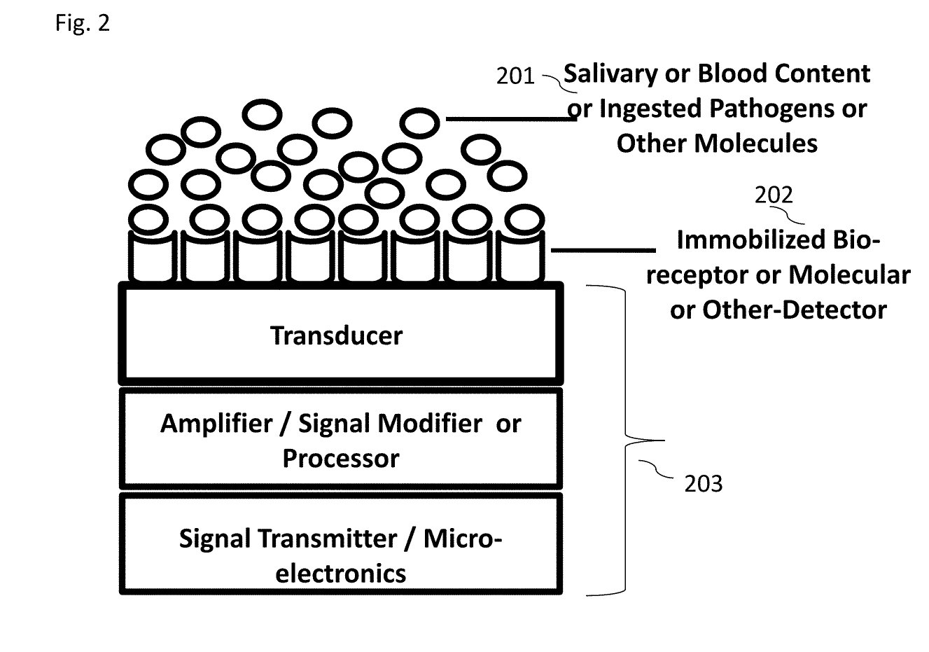

FIG. 2 exemplifies the structure of a sensor or biosensor 201 as may be used in embodiments of the invention. At the top, a biologically active or non-inert surface contains, e.g., biosensors—bio-detectors, bio-receptors, or biologically sensitive materials, and is exposed to biomarker molecules 201 (see, e.g., Table I). Bio-detectors, bio-receptors, or biologically sensitive materials 203 include, but are not limited to, biologically-derived materials, bio-mimics, and chemical and bioengineered molecules. The sensors interact with biological molecules or biomarkers, and this recognition is converted by transducers 202 to a signal that is more readily measured and quantified. A transduced signal is transferred to a signal amplifier or modifier, which transfers the signal to a wireless transmitter in this embodiment. These biosensors could be custom-made by 3D printing.

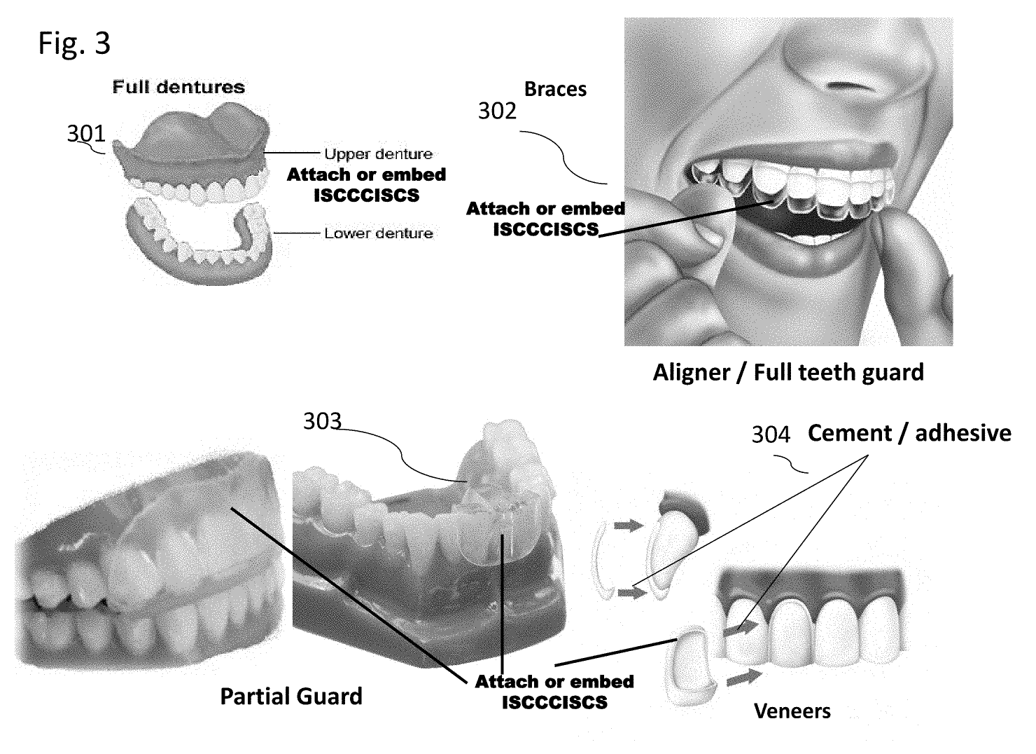

FIG. 3. Unfixed dental devices are defined as ones not permanently attached to the jaw bone, but as possibly attached to the gum or teeth. Temporary ISCCCISCS mouth guards 603 generally have a shortened life-span compared to fixed devices, but they may be placed in the oral cavity for from several minutes to several months (but typically are not designed for placement, e.g., for several years). ISCCCISCS are optionally attached to or embedded in these devices. Some examples of unfixed dental devices are partial and full dentures, braces, retainers, full or partial veneers, full or partial aligners, and guards 301-306. These ISCCCISCS could be custom-made by 3D printing.

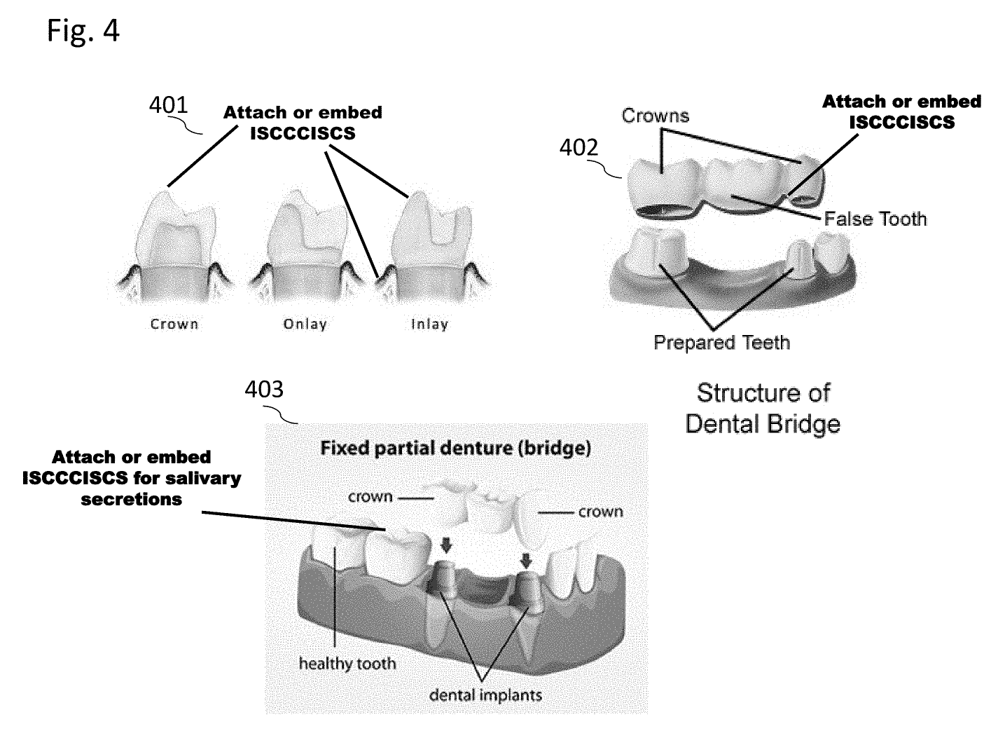

FIG. 4. Fixed or permanent dental devices: In certain embodiments, these dental devices are attached to the jaw bone through metal, dental enamel, etc. inserts. Fixed partial denture devices and ISCCCISCS contained or inserted therein may be attached permanently and placed in the oral cavity for several years. This provides a unique opportunity to place ISCCCISCS in gums with plenty of blood supply or in jaw bones. Patients with bone disease or who are at risk for bone disease such as osteoporosis will particularly benefit from biosensors that are capable of determining and assessing bone health. ISCCCISCS may also be attached or embedded into these devices so that the ISCCCISCS are exposed to secretions in the oral cavity. Examples of such dental devices are crowns, onlays, inlays, bridges, and dental implants 401-406.

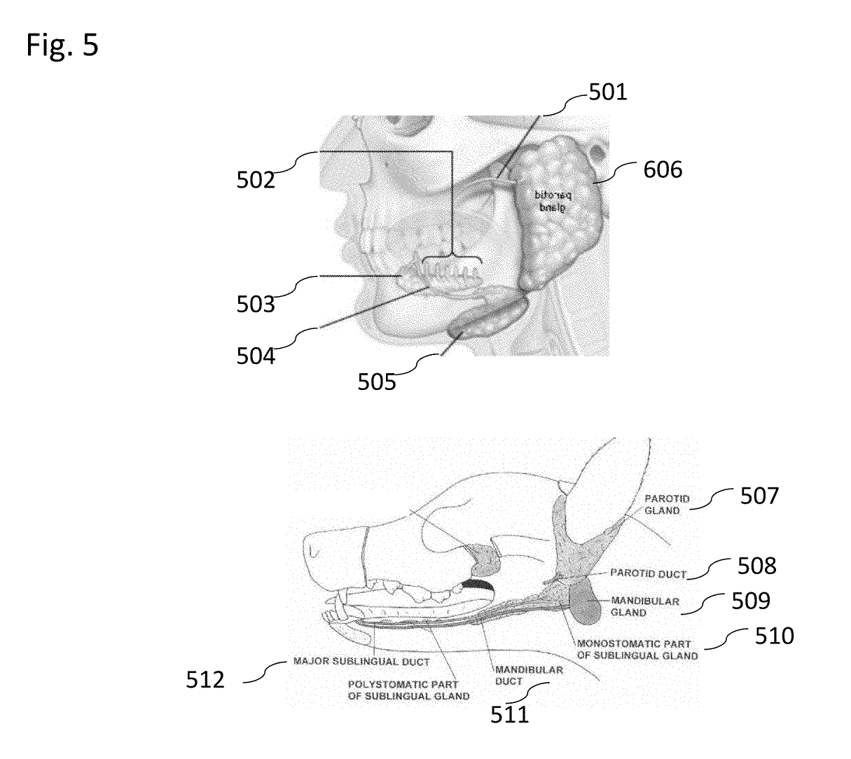

FIG. 5 depicts location of various salivary glands and their ducts. In humans and other mammalians such as cats, dogs, and horses the salivary glands consist of three major pairs of larger salivary glands, which are the parotid glands 506, 507, submandibular glands 504, 510, and the sublingual glands 503, 510, 512. and hundreds of small glands which are found in the mucosal tissues lining the oral cavity. The parotid glands are flattened almond size glands located just beneath the dermis, in front of and below the ear 506, 507. A duct extends to an opening in the oral vestibule near the base of the second molar 501. The submandibular glands have an oblong shape and are present just inside the back corners of mandible 504, 511. Ducts from each submandibular gland open in floor of the mouth in front of the base of the tongue 504, 509. The small elongate sublingual glands are located beneath the tongue 503. There are multiple ducts extending from the sublingual glands to the floor of the mouth beneath the tongue 502, 512.

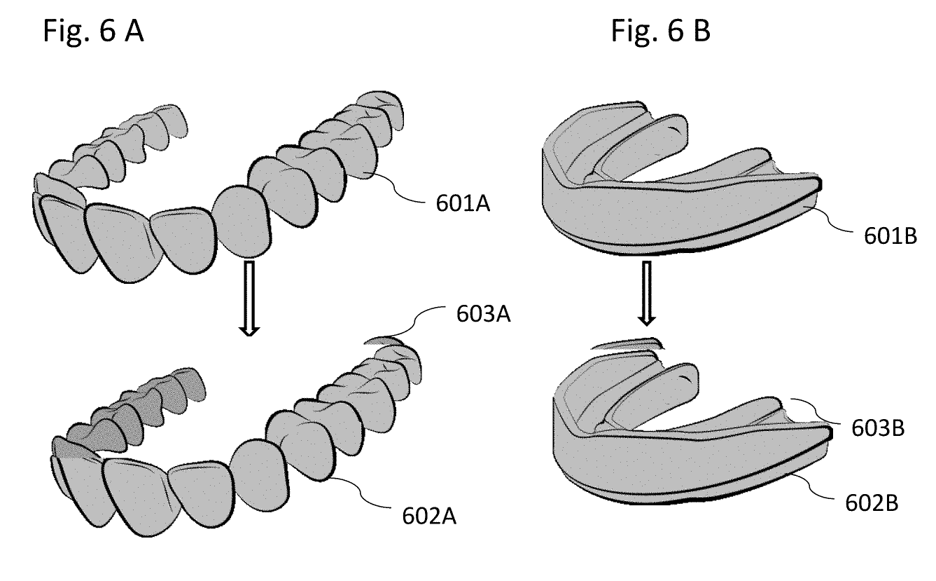

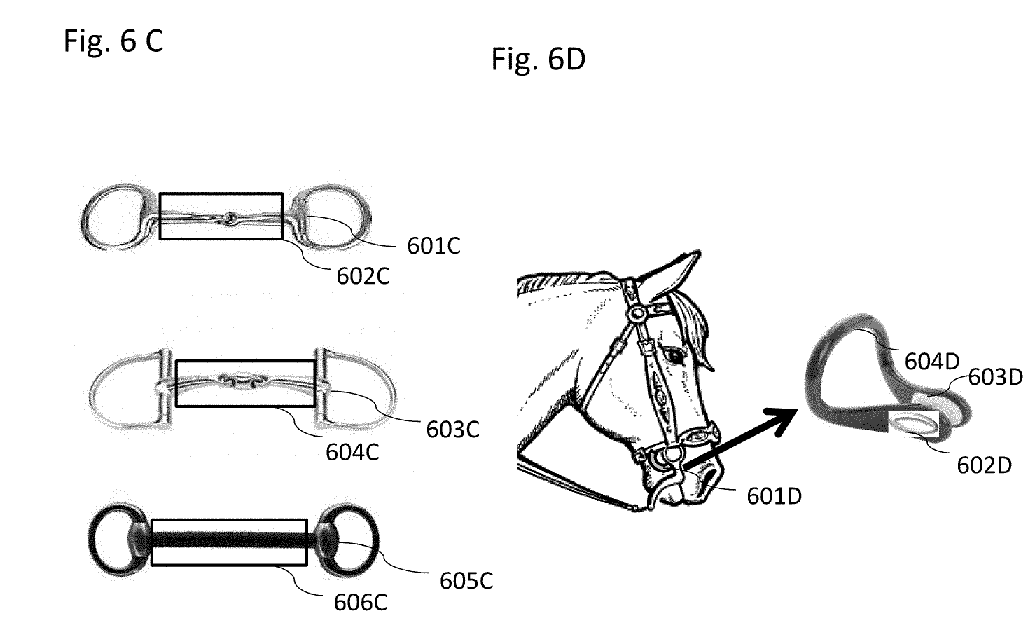

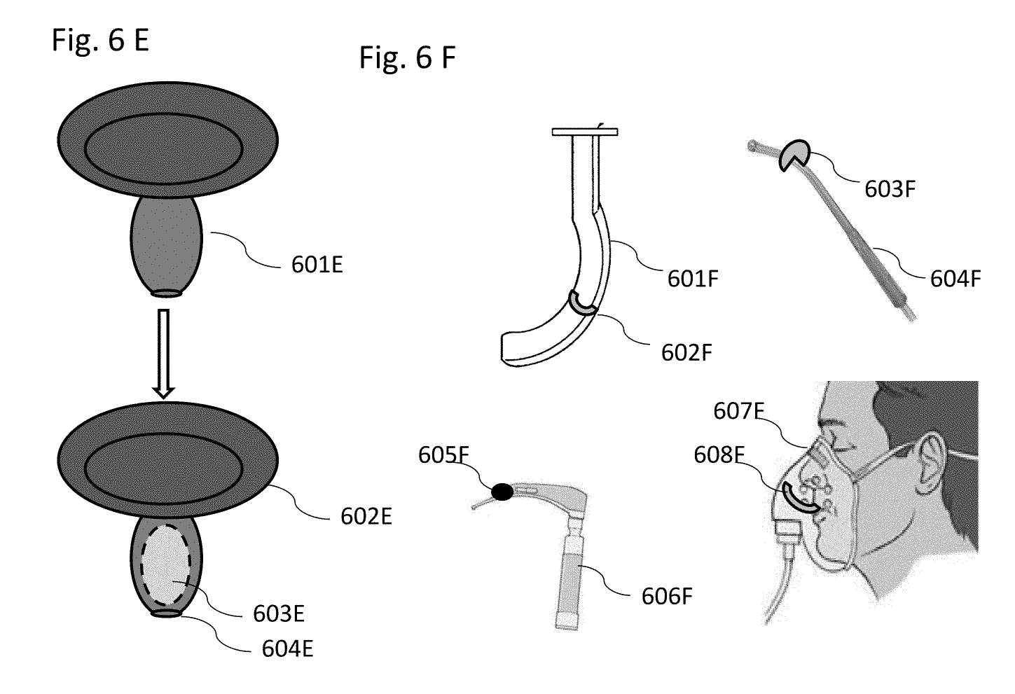

FIG. 6A depicts embodiments which exemplify the locations of ISCCCISCS on a retainer. The placement of ISCCCISCS on retainer or any other dental device would be determined by several factors including, but not limited to, anatomy and location of the salivary glands as depicted in FIG. 5, biologic to be collected, concentrate, and detected, production and manufacturing requirements and ease, patient comfort and convenience, and regulations. It is understood by anyone familiar with the arts that more than one embodiment is possible. However, depicted herein is an embodiment where the ISCCCISCS is located on the inner side of lower jaw retainer 603A; 601A, 602A depicts front of the retainer, which is unchanged. This location is near submandibular and sublingual duct openings, which would assist in fast diffusion and thus the fast concentration of biologics from these glands. The ISCCCISCS could be permanently or temporarily attached to the retainer, and the temporary attachment could be detachable such that the user or caretaker could attach or de-attach it.

FIG. 6B depicts embodiments which exemplify the locations of ISCCCISCS on a mouth guard. The placement of ISCCCISCS on the mouth guard or any other dental device would be determined by several factors including, but not limited to, anatomy and location of the salivary glands as depicted in FIG. 6A, biologic to be collected, concentrate, and detected, production and manufacturing requirements and ease, patient comfort and convenience, and regulations. It is understood by anyone familiar with the arts that several embodiments are possible. However, depicted herein is an embodiment where the ISCCCISCS is located on the inner side of upper jaw mouth guard 603B; 601B, 602B depicts front of the mouth guard, which is unchanged. This location is near parotid glands duct openings, which would assist in fast diffusion and thus the fast concentration of biologics from this gland. The ISCCCISCS could be permanently or temporarily attached to the mouth guard, and the temporary attachment could be detachable such that the user or caretaker could attach or de-attach it. Some or all parts of the ISCCCISCS device could be made of edible parts.

FIG. 6C depicts embodiments which exemplify the locations of ISCCCISCS on a horse-bit. The placement of ISCCCISCS on horse bit or any other dental device would be determined by several factors including, but not limited to, anatomy and location of the salivary glands as depicted in FIG. 3, biologic to be collected, concentrate, and detected, production, development, and manufacturing requirements and ease, patient, human or animal, comfort and convenience, and regulations. It is understood by anyone familiar with the arts that more than one embodiment is possible. However, depicted herein is an embodiment where the ISCCCISCS is placed inside of the horse-bit. The ISCCCISCS is exposed to saliva when the saliva enters the holes as depicted 602C; 604C, 606C. The horse-bit can of different types, not jointed, single jointed, or double jointed. The concentrated saliva, ISCCCISCS, or biosensors can be removed from the side 601C; 603C, 605C. This location primarily based on structure of the horse-bit and not on proximity of glands

FIG. 6D depicts embodiments which exemplify the locations of ISCCCISCS on a cheek or lip clips. The placement of ISCCCISCS on the cheek or lip clip, which could be replaced by inner cheek or inner lip patches, or any other dental device would be determined by several factors including, but not limited to, anatomy and location of the salivary glands as depicted in FIG. 3A, biologic to be collected, concentrate, and detected, development, production and manufacturing requirements and ease, patient comfort and convenience, and regulations. It is understood by anyone familiar with the arts that several embodiments in humans and animals are possible. However, depicted herein is an embodiment where the ISCCCISCS is located on the inside mouth with the ISCCCISCS module facing oral cavity 602D. The surfaces facing oral tissue inside mouth could contain sensors for measuring vital signs, tracking, location, etc. 603D. Loop connects inside of the device with outside 604D. Both inner surfaces that is inside and outside of mouth could contain glue, cement, adhesive, suction cups, etc. 603D to ensure that the device is securely attached 603D; furthermore, the outside of the device could be attached to equine equipment such as bridle, chin strap, etc. to secure the device and prevent swallowing through the loop or outside surfaces of the mouth 601D. The ISCCCISCS could be permanently or temporarily attached to this device, and the temporary attachment could be detachable such that the user or caretaker could attach or de-attach it. This device could be superior to horse-bit because the horse bit is subject to tongue motion artifacts which could be lacking in this device. Also, this device could have better contact with tissue for accurate measurements. Batteries could be placed either inside or outside of the oral cavity.

FIG. 6E depicts embodiments which exemplify the locations of ISCCCISCS on a pacifier. The placement of ISCCCISCS in/on the pacifier or any other dental device would be determined by several factors including, but not limited to, anatomy and location of the salivary glands as depicted in FIG. 3, biologic to be collected, concentrate, and detected, development, production and manufacturing requirements and ease, patient comfort and convenience, and regulations. It is understood by anyone familiar with the arts that several embodiments in humans and animals are possible. However, depicted herein is an embodiment where the ISCCCISCS is located on the inner side of the pacifier mouthpiece 604E. Also, depicted here is parts of pacifier that remain inside, mouthpiece, 603E and outside of the mouth 601E, 602E. The ISCCCISCS could be permanently or temporarily attached to the mouthpiece and the temporary attachment could be detachable such that the user or caretaker could attach or de-attach it through parts of the out of the mouth 601E, 602E.

FIG. 6F depicts embodiments which exemplify the locations of ISCCCISCS on medical equipment. The placement of ISCCCISCS in/on the medical equipment or any other dental device would be determined by several factors including, but not limited to, anatomy and location of the salivary glands as depicted in FIG. 3, biologic to be collected, concentrate, and detected, development, production and manufacturing requirements and ease, patient comfort and convenience, and regulations. It is understood by anyone familiar with the arts that several embodiments in humans and animals are possible. However, depicted herein is an embodiment where the ISCCCISCS is located on the outside 602F, 603F, 605F, 608F of the guedel oral airways, oral Suction catheter, Laryngoscope, and oxygen mask 601F, 604F, 606F. The ISCCCISCS could be permanently or temporarily attached to these devices, and the temporary attachment could be detachable such that the user, physician, or caretaker could attach or de-attach it.

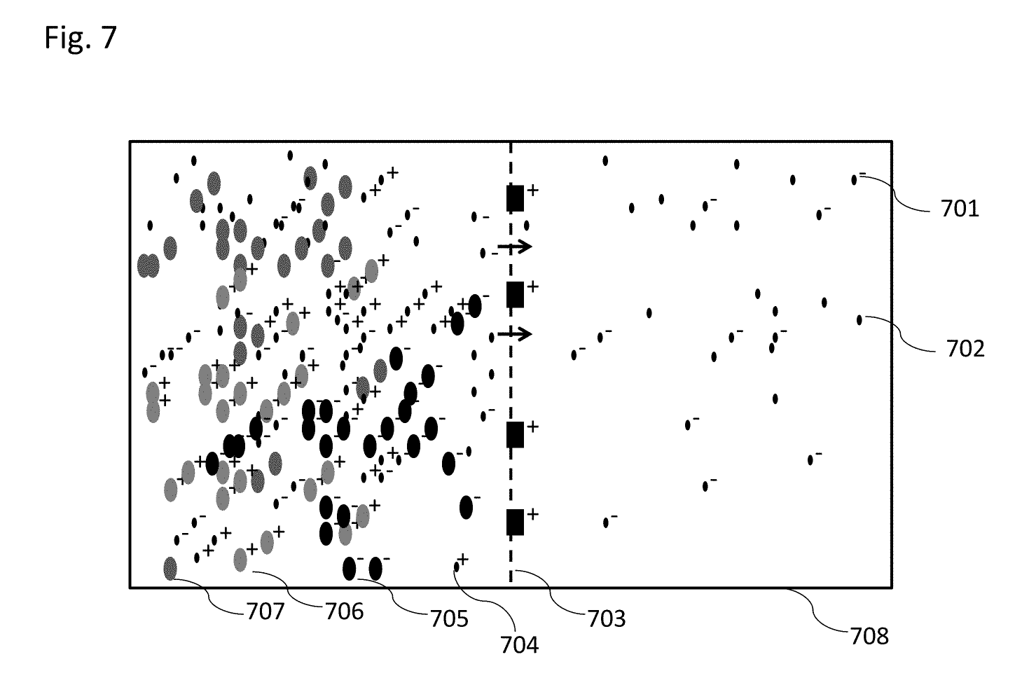

FIG. 7 depicts embodiments of ISCCCISCS which exemplify neutral and charged molecular movement across a semi-permeable membrane in the present invention. It is understood by anyone familiar with the arts that several embodiments are possible with membranes of several different characteristics as explain before. Furthermore, the membrane could be replaced by other materials such as, but not limited to, micro- or nano-coated or uncoated beads, film, gel, and others materials. The purpose of these membranes or micro-beads is to separate components of salivary and oral fluids in-situ. In this embodiment of the invention, the semi-permeable membrane could separate salivary components by size and charge 703. In this example, six different types of molecules are present, small negatively charged 701, neutral small 702, positively charged small 704, negatively charged large 705, positively charged large 706, and neutral large 707. The container is impermeable to any molecule 708. The membrane here has a positive charge such that only negatively charged molecules or moieties could cross it 703. Moreover, any large molecule could not cross the membrane due to smaller pore size. The movement of the small neutral and small negatively charged molecules would occur as determined by electrochemical gradient and would stop when there is no electrochemical gradient. Not shown here, but in an embodiment of this invention, respired airs could add or replace saliva to measure and analyze respiration.

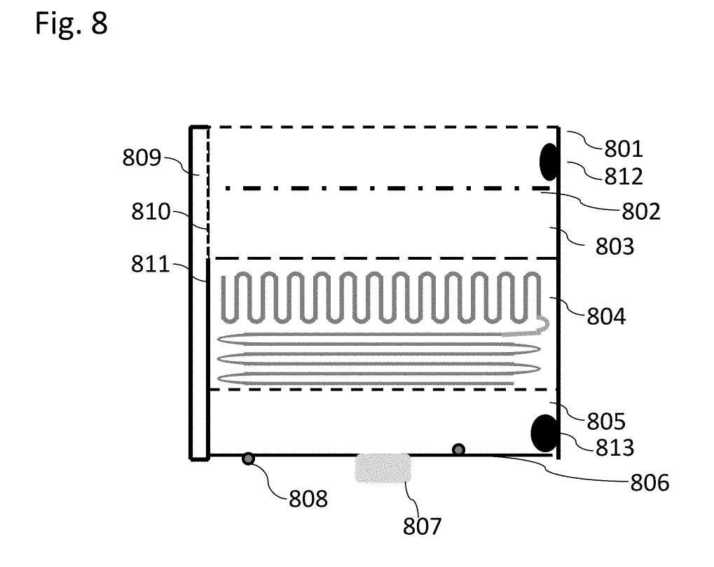

FIG. 8 depicts embodiments which exemplify collection, concentration, isolation, storage, and detection module, chip, or cartilage of the salivary fluids. The embodiments presented in this patent application aim to in-situ concentrate desired salivary components through separation and isolating the desired components from each other so that the desired components become accurately detectable. Several salivary components cannot be accurately detected and in-situ because they are present at sub-detection levels and due to hindrance from food debris, plaque, and other large particulate contaminants in the oral cavity or interference from other salivary components. It is understood by anyone familiar with the arts that several different arrangements of membranes, micro-beads, microfluidics, micro-pump, micro-suction pumps, etc. for collection, concentration, storage, and detection module, chip, or cartilage embodiments are possible and could depend upon biologic to be collected, isolated, concentrated, and detected. The regulation and requirement for device production manufacturing, patient comfort, ease of use, and convenience would determine the ultimate device design. Saliva could enter the entry chamber across a size exclusion semi-permeable membrane, which could remove food debris, plaque, etc. 801. The entry chamber would be in close proximity to salivary glands. In some embodiments, although not shown here, the entry chamber would be connected to a trans-dermal extraction module, which is in contact oral tissues. Suction micro-pump can enhance this entry 812. Saliva collected in the entry chamber could be further concentrated by two different techniques, first through a hydrophilic membrane or micro-beads 810 into a water collecting chamber 809. Because different amounts of saliva could be produced by individuals at different times and different saliva could be produced by different individuals, this water collecting chamber along with the entry chamber could provide information about the amount of saliva produced. These chambers could be used to calibrate saliva and normalize the data collected and could also measure both amounts and rates of saliva production in any oral cavity. Not shown here, this chamber could be connected to or contain sensors, such as, but not limited to, pressure and touch-sensitive sensors, to measure and quantify collected saliva. This chamber could also contain chemicals coated surfaces, chemicals, or reactive sensors that change color as saliva interacts with them. Then assist in visualization of collected data for proper calibration and normalization. In one embodiment of this could be when pH, due to changes in electrolyte and oral hydration, of oral fluids changes, the color of an athletes' mouth guard could change for visual results or measured by optical or photo sensors. This change in pH could also be used to measure oral infections or propensity of infection. Second through another semi-permeable membrane which could be ionic or charged membranes, size-exclusion membranes, affinity membranes, adsorption membranes, extracting membranes, hydrophilic membranes, hydrophobic membranes, electrostatic attraction and repulsing membranes, lipophilic membranes, lipophobic membranes, reactive membranes, etc. 802. Salivary and oral fluid components that enter the collecting chamber 803 could then concentrating module or cartridge, microfluid chip 804. Concentrated saliva could enter further be concentrated by milli-fluidics module, microfluidics module, or nanofluidics chip or module; paper microfluidics could be utilized in this embodiments. Final concentrated saliva could into concentrate collecting chamber 805. This final concentrate could be either exposed to right-sided biosensor 806 or could be removed for further processing and detection through concentrate remove opening 807. The module could also contain outside biosensor or sensors for detecting non-saliva biological vitals such as core body temperature, heart rate, etc. 808. One or more micro-pump could be placed in any of the chambers that could assist in controlled manner suction of saliva or its' components into various chambers 812, 813.

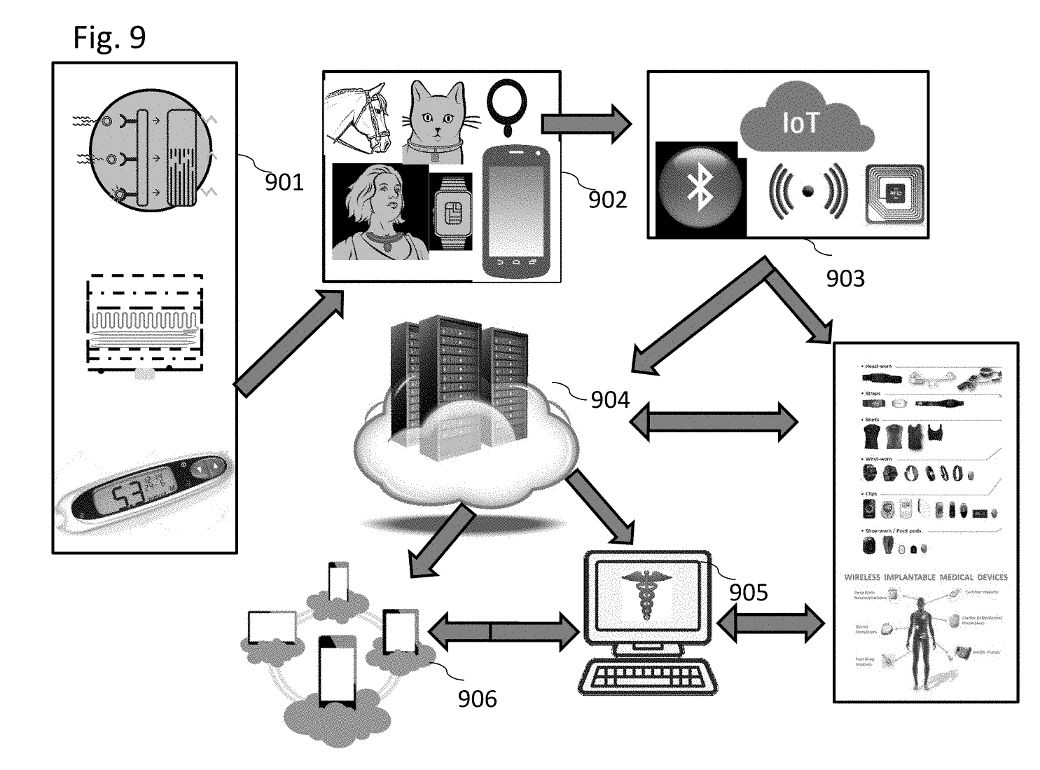

FIG. 9 depicts embodiments which exemplify ISCCCISCS dynamic alerting software and secure networks, in accordance with embodiments of the present invention. In these embodiments of the described ISCCCISCS, an example of a fully integrated diagnostic and performance measurement system is provided. 904 represent a secure host server or cloud networks which includes all forms of smart devices, one or more pagers, SMS, Faxes, emails, GIS mappers, beacons (XYZ) telephones, PSTN devices (Voicemail, IVR, ASR, TTS), satellite phones and other forms of communication which can be implemented and utilized by one or more individuals, one or more animals, or one or more organizations 905, 906. Also, the present invention can include a privatized internal server host and subsystems as well as one or more external hosted alert servers. A plurality of collective data can be derived from several ISCCCISCS oral measurements including, but not limited to, the integration of any wearable, sensors, and medical devices such as implantable medical devices, on body sensors and wearable and environmental sensors and devices 907 and other embodiments in the present invention. A plurality of sensor and biosensor data from ISCCCISCS and ISCCCISRS could inform all smart devices through a variety of wireless technologies such as Wi-Fi, Wi-Fi Direct, Bluetooth, ZigBee, Internet of Things, Internet of Things, MiFi 901, 902, 903. All wearable devices, whether smart or not smart, and also all RFID readers can be examined and analyzed to determine the degree of an alert (low, medium or high). The alerts would be dispatched through various templates referred to as friends and family which includes trainers, caretakers, and other authorized personnel, and electronic medical records 905, 906.

In humans, oral fluid originates mainly from secretions of the parotid, sublingual, and submandibular glands, and a large number of minor salivary glands. Non-glandular constituents of salivary fluids originated from esophageal mucosa, food debris, crevicular fluid, blood-derived compounds, infections, and many other oral bio-sensate molecules could potentially be used in diagnosing health, disease status, sub-optimum health, and performance. Additionally, the collection and evaluation of secretions from individual salivary glands are used for the detection of gland-specific pathologies such as infection and obstruction. The Academy of General Dentistry suggests that more than 90 percent of all systemic diseases, diseases that affect or pertain to the entire body and not just one of its parts, produce oral symptoms and are reflected in oral secretions. Thus, the oral cavity serves as a critical vantage point for detecting the early onset diseases including, but not limited to, systemic infections like AIDS, cardiovascular diseases, atherosclerotic inflammation, Cushing disease, metabolic diseases, stress, electrolyte imbalance, stroke, preterm and low-birth-weight delivery, dental diseases, tooth decay, and other diseases. It is a site for oral cavity infections that damage teeth and gums. In the case of humans, the elderly, babies, and the disabled who need constant monitoring but are unable to communicate their health status could also benefit from real-time detection of such biosensors. The “Disposable Medical Devices Sensors Market” includes, but is not limited to, the biosensor, blood chemistry, and salivary analysis, saliva collection by spitting or other non-in-situ means, a diagnostic capsule endoscope, blood glucose strip, therapeutic insulin pump, and global diagnostic forecast. However, whole saliva systems are not available for real-time study and evaluation of systemic disorders, and there is a lack of an effective means for real-time diagnosis. One major reason for this is very dilute salivary components, which makes detection difficult especially for real-time analysis. Accurate measurements of diagnostic and performance parameters via oral cavity are problematic due to interference from oral components such as mucosal cells, food debris, plaques, microbes, etc. The invention presented here would resolve this significant problem.

These aforementioned commonalities are true for animals as it is true for human and related animal diseases (and see some examples of, in Table I):

TABLE I
Disease

| | |
|---|---|
| | Biologically derived Biomarkers |
| Cytomegalovirus infection | Nucleic Acid |
| Dengue viral fever | IgA (immunoglobulin), Elevated Body Temperature |
| Ebola virus infection | IgG (immunoglobulin), Nucleic Acid, and Elevated Core Body |
| | Temperature |
| Multiple Sclerosis | IgA (immunoglobulin) |
| Epstein-Barr virus infection | Nucleic Acid and Proteins |
| Herpes simplex viral infection | Proteins, and Nucleic Acid |
| Acquired Immune Deficiency | IgG, (immunoglobulin), Nucleic acid, Blood Chemical, |
| Syndrome | Microbes, and Protein |
| Hepatitis A viral infection | IgM, IgA, IgG, (immunoglobulin), Proteins, Microbes, Nucleic |
| | Acid |
| Hepatitis B viral infection | HbsAg, HbsAb, HbcAb, (immunoglobulin), Proteins, |
| | Microbes, and Nucleic Acid |
| Hepatitis C viral infection | IgG, (immunoglobulin), Proteins, Microbes, and Nucleic Acid |
| Human herpesvirus infection | Nucleic Acid, Proteins, Microbes, and IgG |
| Malaria, Plasmodium | IgG (immunoglobulin), Elevated Core Body Temperature, |
| falciparum infection | Microbes, Nucleic acids, Proteins |
| Periodontal disease | Protein, Microbes, Gases, and Nucleic acid |
| Oral and Lung Cancer | Protein, Microbes, Gases, and Nucleic acid |
| Mammary & Pancreas Gland | Protein, Microbes, Gases, and Nucleic acid |
| Carcinoma | |
| Mycobacterium tuberculosis | DNA (Nucleic Acid), Elevated Core Body Temperature, |
| infection | Protein, Microbes, Gases, Nucleic acid |
| Age-Related Diseases | Methylated DNA (modified Nucleic Acid), Nucleic Acids, |
| | Proteins, Modified Proteins, Blood Chemicals, Gait, |
| | Movement, Immunoglobin |
| Sjogren's syndrome | Nucleic Acid and Protein |
| Oxidative stress | Protein and Nucleic Acid |
| Inflammation | Protein, Elevated Temperature, Microbes, Nucleic Acid |
| Malnutrition | Proteins, Cations, Anions, Micronutrients, Vitamins, Minerals |
| Allergies | IgG (immunoglobulin), Allergens, Microbes, Nucleic Acid |
| Genetic Disorders, Cystic | Nucleic Acid, Proteins, Salts, Cations, and Anions like K⁺, Cl⁻, |
| Fibrosis | NaCl, Ca²⁺ |
| Blood Disorders | Immunoglobulins, Proteins, DNA, Microbes, Blood Chemical, |
| | and Nucleic Acids |
| Cardiovascular and | Proteins, Immunoglobulins, Microbes, Blood Chemical, Blood |
| Circulatory Diseases | Pressure, and Nucleic Acids |
| Renal Disease and Urinary | Proteins, Nucleic Acid, Salts, Cations, Anions, Minerals, |
| system Diseases | Ammonia Containing Gases, and Blood Chemicals, urea, uric |
| | acid |
| Osteoporosis, Skeletal system | Proteins, Minerals, Salts, Nucleic Acids, Electrical Waves, |
| diseases, and Muscular | Blood Chemicals, Immunoglobins, and Microbes |
| diseases | |
| Integumentary system diseases | Proteins, RNA, DNA, Electrical Waves, Salts, Blood |
| | Chemicals, Immunoglobins, Blood Chemical, and Pathogens |
| Fatigue | Proteins, Lactose, Metabolites, Nucleic Acids, and blood |
| | Chemicals |
| Stress | Cortisol, Immunoglobulins, Proteins, Microbes, Blood |
| | Chemical, and Nucleic Acids |
| Crushing syndrome | Cortisol |
| Taste, Ageusia, Hypogeusia, | Neuro-peptides, Neuro-chemical, Nerve function, Electrical |
| Dysgeusia | Properties, Gases, Microbes, Protein, Nucleic Acids, Chemicals |
| Chewing, Mastication | Movement, Proteins and Digestive Enzymes, Pressure, DNA, |
| | and RNA |
| Eating disorders | Regurgitated food, digested or partially digested food, stomach |
| | content, stomach acid, and cells. |
| Bad or Fruity Breath | Neuro-peptides, Neuro-chemical, Nerve function, Electrical |
| | Properties, Gases, Microbes, Protein, Nucleic Acids, Chemical |
| Digestive System, Liver, | Proteins, RNA, DNA, Enzymes, Electrical Waves, Blood |
| Spleen, Gall-bladder, Pancreas | Chemical, Chemicals, Immunoglobins, Pigments, Acids, Salts, |
| | Anions, Cations, PH, and Pathogens |
| Endocrine, Lymphatic, and | Proteins, RNA, DNA, Electrical Waves, Chemicals, |
| Excretory Diseases | Immunoglobins, Blood Chemical, Pathogens, Salts, Hormones, |
| | and Lymph Fluids |
| Sleep Deprivation | Alpha-Amylase, Sleep Patterns, Motion, Neuro-peptides, |
| | Neuro-Chemical, Nerve function, Electrical Properties, Gases, |
| | Protein, Nucleic Acids, Chemicals, and Breathing |
| Bruxism | Pressure, contact, touch sensors. |
| Stroke | Muscle tone, Electromyography (EMG), Proteins, and Nucleic |
| | acid |
| Fertility, Ovulation, | Proteins, Hormones, Nucleic Acids, and Core Body |
| Reproductive Diseases | Temperature |
| Depression, Alzheimer's | Proteins, RNA, DNA, Electrical Waves, Chemicals, |
| Disease, Neurological | Immunoglobins, Blood Chemical, Neuro-Peptides, Neuro- |
| Disorders, Dementia and other | Chemical, Nerve function, Electrical Properties, Gases, |
| Neuro-Diseases | Microbes, Nucleic Acids, Chemicals and Pathogens |
| Psychology, Mental Disorders | Proteins, RNA, DNA, Electrical Waves, Chemicals, |
| and Mentally Challenged | Immunoglobins, Blood Chemical, Neuro-Peptides, Neuro- |
| | Chemical, Nerve function, Electrical Properties, Gases, |
| | Microbes, Nucleic Acids, Chemicals, Gait, Tracking, |
| | Movement, Behavior, and Behavior-Patterns |
| Nervous system diseases | Proteins, RNA, DNA, Electrical Waves, Chemicals, |
| | Immunoglobins, Blood Chemical, Neuro-Peptides, Neuro- |
| | Chemicals, and Pathogens |
| Diabetes | Sugar, Acetone, Proteins such as hba1c, Proteins, Nucleic acids, |
| | and Blood Chemical |
| Dehydration | Salts, Cation, and Anion Concentration, Humidity or Water |
| | Concentration, Proteins, and Nucleic Acids |
| Halitosis | Sulphur Containing Gases, Microbes, Enzymes, Nucleic Acids, |
| | and Proteins |
| Respiratory and Pulmonary | Immunoglobin, Salts, Water Concentration, Nucleic Acids, |
| Disease | Proteins, Temperature, Blood Chemicals, Gases, Lung Capacity, |
| | and Muscle Tone |
| Ear, Nose, Throat Diseases | Temperature, Pulse, Blood Chemicals, Proteins, DNA, RNA, |
| | Electrolytes, Movement, Gait, Gases |
| Infant and children diseases | Temperature, pulse, blood chemicals, proteins, DNA, RNA, |
| | Electrolytes, Movement, Gait, Gases |
| Alcoholism | Alcohol, Proteins, Enzymes, and Nucleic Acids |
| Pollution | Chemicals, Gases, Proteins, Nucleic Acids, Microbes, and |
| | Pollutants |
| Drug addiction | Drug molecules, Proteins, and Nucleic Acids |
| Allergies | Allergenic Molecules, Immunoglobins, Nucleic Acid, and |
| | Proteins |
| Hypoxia | Decreased Oxygen Concentration, Blood Chemicals, Proteins, |
| | Nucleic Acid, Gases, and Pressure |
| Toxins | Gases, liquids, Solids, Proteins, Blood Chemicals, and Nucleic |
| | Acids |
| Smoking | Nicotine, Proteins, and Nucleic Acids |
| Gait | Biological Biosensors: Nucleic Acids, Proteins, and Blood |
| | Chemicals |
| | Non-Biological Sensors: Accelerometer, Gyroscope, Inertia |
| | Sensors, |
| Obesity | Selenomonasnoxia, Proteins, Nucleic Acids, and Blood |
| | Chemical |
| | Microbial Biosensors |
| Cancer, oral | Capnocytophagagingivalis, Prevotellamelaninogenica, |
| | Streptococcus |
| Cancer, Pancreatic | Neisseria elongata, Streptococcus mitis |
| Crohn's disease | Fusobacteria, Firmicutes |
| Chronic pancreatitis | Granulicatellaadiacens, Streptococcus mitis |
| Periodontal disease | Aggregatibacteractinomycetemcomitans, |
| | Porphyromonasgingivalis, Prevotella intermedia, Tannerella |
| | forsythia Campylobacter rectus |
| Dental Caries | Streptococcus mutans, Lactobacillus |
| Obesity | Selenomonasnoxia, proteins, nucleic acids, blood chemical |

The presence of various disease-signaling salivary biomarkers permits accurate reflection of normal and disease states in animals and humans. Information derived from the oral cavity is capable of augmenting or possibly replacing blood sampling. Oral cavity information may also be used as an efficient precursor before other more invasive medical diagnostics are employed. This is possible because saliva represents blood components in both composition and relative concentration. However, a large number of components are present at concentrations below detectable levels. Therefore, currently available methods for the detection of such salivary biomarkers are inefficient for in-situ rapid detection and analysis. Furthermore, they do not alert or communicate information derived from the collection, isolation, and/or detection of the concentrated biomarkers that are contained in the saliva. Current usage of the salivary biomarker is gathered via rudimentary swaps or spitting, and the sample is then sent to a laboratory for individual component isolation, detection, and analysis. Therefore, information is not available at point of care and has limited utility in clinical diagnoses. Thus, there exists a critical need for improved devices, methods, and systems in animals and humans for ISCCCISCS and detection technology in real or near-time. The significance of its use corresponds to physiological and health information gathering, assessment, monitoring, and ultimately, health care, therapeutic, and performance assistance.

Additionally, currently there is a profound lack of integration between a multitude of cross-linked technologies and skills when information is gathered from saliva. As this information is regarded concerning metadata diagnosis, in-situ collection and concentration of oral fluid components by biochemical and biophysical technologies, real-time detection, analytics, multimedia, communication networks, and alerting; all of these would enhance health and performance. Also, these current limitations restrict a multi-dimensional approach which could seamlessly measure related physiological parameters, health, and performance of individuals and animals with greater accuracy, convenience, yet far less intrusively. The lack of integration between disciplines fails to address the growing need for the next level of metadata, biochemical, biophysical, milli-fluidics, microfluidics, nanofluidics, and biological tools which could provide early detection of sub-optimum health and performance, diseases, and medical disorders with greater accuracy as stated herein in the present invention.

Similar to all solutions, saliva is made up of solute and solvent. The saliva as solution contains a large percentage of water as a solvent and many solutes such as several biologics that are present in blood, food debris, ingested pathogens, ingested toxins, ingested allergens, ingested drugs, ingested nutrients, ingested food, plaque, plaque-related microbes, mucosal cells, and/or mucosal-cell-debris. Solutes in all solutions can be concentrated and isolated by use of thermodynamic principles and use of semi-permeable membranes, which allows one fluid component to cross the semi-permeable membrane compared to other components. When semipermeable membranes separate two solutions of different solvent concentration, the solutes diffuse from the high concentration region to the low concentration region. This flow of solvent constitutes osmotic flow or osmosis. Therefore, through osmosis, water is pulled from an area of lower solute concentration to an area of higher solute concentration. This osmosis is as true for chemical solvents as it is true for electrically charged molecules. The concentration gradients of chemical and electrically charged molecules across semi-permeable membranes constitute an electrochemical gradient.

Recently, 1, 2, or 3 D milli-fluidics, micro-fluidics, and nanofluidics are used to isolate biological components from biological samples for diagnostics. An example of a micro-fluidics technique is a micro-fluidic chip containing a set of micro-channels etched or molded into a material to isolate different proteins or DNAs. To consider milli-fluidics, micro-fluidics, and nano-fluidics, at least one dimension of the channel must be in the range of a millimeter, micrometer, or nanometer. Properties of fluid or gas flow are different between macro-fluidics, milli-fluidics, micro-fluidics, and nanofluidics.

The ISCCCISCS can be used to quickly, in real-time, and/or continuously monitor and diagnose at point-of-care, e.g., clinic, hospital, hospice, operating room, etc. The ISCCCISCS can be attached to a variety of medical equipment and devices. These medical equipment and devices can, include stretch stripes, adhesive stripes, oral surgical, medical devices, hospitalization equipment, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, Eschmann stylet or gum elastic bougie, respirator, mucus sucker, and etc.

Besides, adult humans, animals such as dog and cats pets and horse and human children can benefit from ISCCCISCS. The ISCCCISCS can be attached to pacifiers for animals and children or to horse-bit, cheek-clip, and lip-clip for horses. The mode of attachment for above-mentioned medical equipment, devices, and child and animal devices can be insertable”, “implantable”, “attachable”, “fittable”, “embeddable”, “temporarily insertable” “permanently insertable”, “temporarily implantable”, “permanently implantable”, “permanently attachable”, “temporarily attachable”, “temporarily fitable”, “permanently fitable”, “temporarily embeddable” and “permanently embeddable” refer to means of securely inserting and attaching in or to, or fastening a device, such as being adhered to, cemented, affixed or otherwise securely attached to a surface or object.

Currently, saliva is collected by spitting and then sent to a far away laboratory for testing. Because the ISCCCISRS can process saliva samples in the oral cavity by concentrating and isolating biologics, it is possible for saliva to be tested at-point-of-care. In these embodiments, the concentrated and isolated biologics would require processing outside of the oral cavity. The biologic would be detected by a reader when the ISCCCISRS device or a part thereof are containing the isolated sample is inserted or otherwise analyzed by the reader. Still, some concentrated and isolated that require laboratory processing would be sent to a laboratory, e.g., the very low blood concentration of RNA that could diagnose cancer would need to be amplified by PCR before they can be accurately identified. Without ISCCCISRS this RNA would not be easily and accurately identified.

The oral device, collection, concentration, isolation, and storage modules and sensors can be personalized depending upon need. As personalized biosensors or sensors of the invention become available, healthcare costs will decrease and outcomes for multiple diseases easier. As technologies such as nano-technology or 3D printing become more advanced and easily and cheaply available, the ISCCCISCS of the invention may be adapted to provide additional savings in healthcare costs for both humans and animals. Furthermore, the present invention enables biosensors to be uniquely communicated from one or more animals and humans to a plurality of ISCCCISCS systems; thus, adding to the utility of the sensors. The ISCCCISCS system of the invention enables humans to monitor and alert themselves or others and enables animal-ISCCCISCS units to send alerts to humans to monitor and alert their caretakers. They also help to preemptively and accurately diagnose, detect, and monitor, and thus, help in the prevention, prognosis, and risk-assessment of a variety of diseases.

A feature of many embodiments of the ISCCCISCS and methods of the invention is the dynamic nature of the integrative alert components that may be built into the customizable operations. For example, people, individuals, groups, teams, hospitals, organizations, etc., may be alerted on a dynamic basis when one or more or various combinations of, program range settings of various parameters are exceeded. The customizable alert modes may be operated as real-time or near-time slumber to alert, programmed, automatic, manual control, geographic-specific, disease or condition-specific, gender-specific, age-specific, situation-specific, activation-triggered, or woken-up by another sensor, device or remote mode, or any combination of such customized parameters and components. The ISCCCISCS can network with other wearables on and around human or animal. Integrate ISCCCISCS, includes one or more of movement, audio, video, pressure, impact, sensory wearables, etc., which provide feedback to enhance accurate measurement of performance, movement, biometrics, and can add in health-related and physiological inputs as well to optimize health, well-being and/or performance criteria.

The ISCCCISCS of the invention in one embodiment, for example, measures inspired and expired gases, inflammation, metabolism, etc. as a component of the “practiced” oral device insert. A “practiced” oral device insert may include any customized device inserted in an animal or humans' oral cavity and is designed to monitor any metabolic situation such as, e.g., those described in Table I. Three dimension printing applied to the invention makes cost-effective and less costly custom dental devices available and thus makes our ISCCCISCS more cost-effective. Conventional methods are limited by time and location of routine diagnoses like blood chemistry. However, ISCCCISCS of the present invention offers a flexible and unique opportunity to measure these statistics at home, at work, during exercise, or even while sleeping without the need for a skilled person to withdraw blood for further analysis. The device or parts thereof may be present in the oral cavity for a short (few seconds to minutes to few hours) or a long time (several days to months to years) and may be temporary or permanent.

Blood chemical measurements amongst animals and pets are expensive and difficult because the pet is required to be anesthetized by a veterinarian before the blood draw. The invention detailed herein would allow pet owners to measure blood or saliva components and by-pass the need to draw blood.

In a further embodiment of the invention, water or humidity can be concentrated and isolated by absorbing and releasing nano- or micro-beads, which expand and contract when water is absorbed or released, could be coupled to touch or pressure sensible sensors to determine hydration and dehydration levels of the ISCCCISCS device wearer. Several medications and diseases cause dry mouth; some examples are antihistamines, antidepressants, diuretics, nonsteroidal anti-inflammatories, narcotics, dehydration, radiation, diabetes, smoking, using chewing tobacco, hormonal imbalances, mouth breathing, sleep apnea, cystic fibrosis, mumps, and autoimmune. Dehydration is a major cause of fatigue during athletic and sports activities.

In yet another embodiment, the ISCCCISCS of the invention may be used in conjunction with other smart wearables or attachables on one or more individuals creating a measurable team diagnosis. Furthermore, smart wearables or other medical devices may be utilized by a collective group, including in any team sports application, i.e., basketball, soccer, baseball, hockey, swimming, track, football, cricket, gymnastics, and other Olympic or global sports, etc. It is understood that the ISCCCISCS of the invention may measure and diagnose individual performances in sports (singularly measured, analyzed and diagnosed) or these may be measured, analyzed and diagnosed, collectively as a team composed of individual players. The device can be implanted, flexible, adhered, fixed, and/or cemented to the floor of the oral cavity, inside of the cheeks, tongue, and the sublingual cavity and/or affixed in other places within the oral cavity. The device locations within the oral cavity are optionally chosen by the patient or his/her caretaker and/or healthcare provider. The choice of location is one based upon a combination of factors including, but not limited to, convenience, comfort, durability, exposure duration to molecules to be measured, and duration of the device placement in the oral cavity, development requirement, ease of manufacturing, and regulations. The device may be placed by a healthcare provider including, but not limited to, physicians, surgeons, dentists, veterinarians, or healthcare provider assistants, caretakers, and/or patients themselves. The placement is usually made with the assistance of one of the following; for example mild sedation, full sedation, local anesthesia, general anesthesia, or other. Dental devices represent biologically inert surfaces; whereas, other areas of the oral cavity such as the gums or tongue, are biologically active and non-inert. The ISCCCISCS device, therefore, may be placed fully on inert surfaces, active biological surfaces and/or a combination of both. However, the ISCCCISCS module must always be exposed to saliva.

ISCCCISCS materials inserted in the oral cavity as described in the present invention may detach, clip, be alerting chips, etc., and can be constructed for a short period (temporarily) or constructed for a long-term (e.g., permanent) period. All time periods for the plurality of orally inserted and attached devices used for one or more functions of the invention may be deployed. ISCCCISCS devices could use various materials to concentrated and isolate salivary components. These concentrating and isolating materials include, but are not limited to, semi-permeable membranes, permeable membrane, semi-permeable membrane, non-permeable, synthetic membranes, biological membranes, microfiltration membrane, ultra-filtration membranes, nanofiltration membranes, osmotic membranes, reverse osmotic membranes, ionic or charged membranes, size-exclusion membranes, affinity membranes, adsorption membranes, extracting membranes, hydrophilic membranes, hydrophobic membranes, electrostatic attraction and repulsing membranes, lipophilic membranes, lipophobic membranes, and reactive membranes. It is understood by anyone familiar with the arts that the membranes can be replaced by coated, non-coated micro-beads, nano-beads or gel, microfluidics, microfluidics, nanofluidics, biosensors, RFID tags, and any inserted attached or detachable dental device used for humans and animals to obtain data and sensor-derived information. The design and construction of the inserted oral device will vary for each species, disease, individual, groups of people, etc. and is customizable to detect, monitor and/or alert for all types of medical conditions.

In another embodiment of the present invention, the design of the ISCCCISCS is configured to help avoid the possibility of choking and increase comfort, for example; made of smooth (or partially or fully encapsulated or encased, oval, round, rounded or in any shape or size of similar utility) without sharp edges. ISCCCISCS materials used herein are optionally made un-chewable or chewable depending on the species, medical need, and functionality, comfort, sterile, and durability requirements. For pets, the materials can be resistant to breakage when chewed. Also, an alert can be sent when the ISCCCISCS device becomes semi and/or completely detached or travels to other parts of the body, such as the esophagus or stomach. The ISCCCISCS material employed may be designed to dissolve or break apart to prevent choking which could be particularly useful in the case of animals, pets, children, the disabled, and the elderly, and others who cannot communicate that the device was detached and swallowed.

In addition to oral sensors and biosensors, the ISCCCISCS of the invention includes sensors, biosensors, and nano-particles located elsewhere within and/or on an animal or human that interfaces with the ISCCCISCS. These devices also are optionally within a human or animal body, e.g., such as a drug dispensing pump or other. Alternatively, the device may be located on the human or animal's body, such as a cardiac monitor, dog collar, and accessories or other. These devices may read and interface with a variety of diverse biosensors such as blood electrolyte components of the ISCCCISCS with a cardiac monitor and a drug dispersing pump as one example. The ISCCCISCS system may also communicate with nano-particles in any body part.

In a further embodiment, ISCCCISCS includes a plurality of wireless local area network (LAN), wide area network (WAN) controllers providing system-wide wireless local area network (WLAN) functions such as intrusion prevention, Radio frequency (RF) management, encryption, communication integrity, quality of service (QoS) and mobility are included within the scope of the current invention. Additionally, one or more databases, routers, secure servers, controllers, switches, etc., may be utilized when transmitting oral sensor data through WAN/Internet configurations and multiple, independent databases are manually or dynamically combined for specific applications. In yet another aspect, a combination of one or more elements of metabolic alerts is combined with one or more mobile and/or wearable devices to observe, monitor, measure and/or alert any particular metabolic situation. The ISCCCISCS of the invention also customizes each alert to tailor the alert and/or response to the specific needs and to help prevent the loss inputs and alerting that may transpire in real-time or near-time, depending on the severity, sources, some inputs and need.

In a further embodiment, one or more databases, secure servers, or other devices are utilized to store and/or capture data. The database contains data collected from the ISCCCISCS and any data input in the system including but not limited to data used to send alerts; here, data encompasses both data captured and collected from ISCCCISCS and data used to send out alerts. One or more software programs may collect, capture, and store data obtained from any of the ISCCCISCS interfaces. Some of this data will be publicly available and viewable, and some of the data will be only for private viewing and available to the relevant users and healthcare providers. To prevent any loss of data, in certain embodiments, when connectivity is lost, and data cannot be transmitted in real time to a central storage system, data is temporarily stored on the local device and transmitted to a central system after connectivity is restored. These software programs may be responsible for all data related aspects such as, but not limited to, data comparative with historical or previous data of same individual or other, management, characterization, filtering, transformation, sorting, processing, modeling, data mining, queries, browsing, inspecting, investigation, retrieval, integrating, dissemination, qualitative, quantitative, symmetric, asymmetric, normalizing, clustering, correlations, computer derived values and rages, simple or complex mathematical calculations and algorithms, analytics, statistical data, predictive data, integrations, interpretation, exploratory, finding abnormalities, performance, data products, consumer data, server data, visualizing and/or presentation in a variety of platforms. Here, data analysis also means software for that analysis for disease and other diagnosis and analysis for both humans and animals. Thus, this software may supplement or partially and/or fully replace a healthcare provider's input, such as that of a physician, veterinarian, etc., or in a non-medical context. It is understood by anyone familiar with the art that the present invention could lower the cost of health-care, and therefore, insurance companies could cover the cost of these devices for both humans and animals.

Saliva and other oral fluids sampling are relatively straightforward, and the presence of various disease-signaling biomarkers in saliva has meant that it accurately reflects normal and disease states in animals and humans. However, current saliva technology presents some significant disadvantages which are greatly improved upon by this invention. In particular, saliva involves the collection, concentration, isolation, storage, and sample determination, and in this invention, ISCCCISCS are cemented, implanted, fitted, or attached to parts of oral cavity/dental devices or embedded in oral devices. Several biological molecules are present at sub-detection amounts in saliva. At these levels, in certain embodiments, the biosensor captures these sub-detectable molecules from concentrated saliva and, when enough molecules are captured, an alert is sent so that the biosensor with captured bioactive or biologics may be removed for further analysis. On the other hand, the sensors within ISCCCISCS module could detect concentrated and isolated biologic. The ISCCCISCS of the present invention aggregates various wireless communications with the oral saliva collection, concentration, storage, and biosensors and biomarkers to monitor and notify users of one or more medical conditions. The ISCCCISCS includes the integration of fitted oral devices not only equipped with ISCCCISCS along with one or more medical sensors and devices (as previously described) but GPS/GPRS location positioning technology within the cavity of the mouth. Also, the ISCCCISCS includes the ability of the inserted oral sensor to communicate with other GPS/GPRS devices on the subject animal or human, or near the subject animal or human.

In additional embodiments, ISCCCISCS oral biosensors are inserted temporarily or permanently in a multitude of animals. The ISCCCISCS of the invention includes systems and methods which interface and interact with combinations of “smart dental” and related devices. ISCCCISCS biosensors and RFID tags can be attached, embedded, glued, inserted, etc. to a pre-existing oral device. The ISCCCISCS and biosensors can be inserted by themselves into the oral tissue with or without a needle and/or plunger. The ISCCCISCS and biosensor, when needed, can be removed by oneself at home using a piston, pulley, etc. or other devices. The dental device containing the ISCCCISCS and biosensors can be disposable, clip-on, stick-on, adjustable, and/or removable. In individuals without teeth, such as babies, the device can be attached to, e.g., the gums. Patients can install, remove, and care for the ISCCCISCS and biosensors themselves by use of moldable plastics or other moldable materials, adhesive strips, etc. The ISCCCISCS is alerting, monitoring, notification and reporting systems and other functions are configured to communicate through all application programming interfaces, e.g., cloud networks (APIs).

The following are examples of various ISCCCISCS communication mode classifications to which the invention is applicable. The values may be unique to each situation and a particular patient, both human and animal, for each biomarker application. Listed below are examples of embodiments of this invention; the ranges and preset values used here are only used as examples. It is understood by anyone familiar with the art that many more examples are possible with both humans and animals without departing from the scope of the invention. In each embodiment presented here, ISCCCIRS can replace ISCCCISC.

1. Real-Time or Near-Time Slumber to Alerts Mode.

In this mode, diagnostic data is collected in real-time, or near-time and alerts are sent based on deviations from the set and/or preset values and/or ranges determined by a healthcare provider. Real-time alerts are only possible with ISCCCISC and not by ISCCCIRS.

Several life-saving drugs may cause severe side effects, e.g., many cancer treatment drugs can cause leukopenia, a condition in which white blood cells (WBC) are dramatically reduced. WBC has a normal range of 4.5K to 10K per microliter. Because WBCs are important in the prevention of infections, leukopenia may cause severe infections and even death. Several cancer treatment drugs also can cause kidney failure, which can cause edema, increased blood pressure, electrolyte imbalance and, perhaps, even death. In an embodiment, cancer drug dispensers may be set to interface with WBC- and Na⁺-saliva and drug ISCCCISC, biosensors. When WBC levels fall below a preset value, e.g., significantly lower than 4.5K per microliter, and Na+ increases above 145 mEq/L, the dispenser pump stops or reduces the amount of the cancer drug dispensed until the WBC levels reach 4.5K per microliter and Na⁺ is around 135 mEq/L. However, if the drug is not administered by a pump, but is dispensed as an oral pill, alerts may be sent to healthcare providers who would recommend the patient to reduce the drug dosage or to completely stop until the WBC and Na⁺ levels reach a normal range. In this embodiment, the ISCCCISCS of the invention may isolate WBC and Na⁺ such that they do not interfere with each other. ISCCCISC may also communicate with both a drug dispenser pump and nano-particles in the medication to determine an exact and accurate blood drug concentration.

The ISCCCISCS of the invention is useful in Cushing disease and disorders, which is occurs in humans, dogs, and horses. Some humans and animals develop pituitary gland tumor which secretes excess and uncontrolled adrenocorticotropic hormone (ACTH). ACTH stimulates production and release of stress hormone cortisol, so much ACTH causes the adrenal glands to make too much cortisol hormone. Unregulated cortisol causes insomnia, weight gain, a slow growth rate in children, bone and muscle weakness, male impotence depression, anxiety, fatigue, headache, etc. In one embodiment of this invention, Quinton, a rescue dog has Hypopituitarism and also developed trauma-related leg injuries. He could be prescribed oral corticosteroid medications in high doses over an extended period to reduce inflammation and pain. However, this could cause insomnia and nightly howling. One cure for this could be to monitor during the evening and nighttime cortisol level frequently. Quinton's owner could use ISCCCISCS pacifier every evening and night to monitor his cortisol and accordingly administer or feed medications such that Quinton could sleep through the night. If required, the veterinarian could get an alert when his cortisol levels deviate and change dosage via drug pump or by calling the care-take or owner.

The ISCCCISCS of the invention is useful in other more complex and life-threatening situations requiring more than a single biosensor. For example, in the treatment of congestive heart failure, cardiac arrhythmia, and post-cardiac surgery heart-attack prevention, digitalis drugs are used. The active compounds in digitalis medications are the cardiac-glycosides. Cardiac-glycosides are the most studied positive ionotropic drugs that increase the force of heart muscle contraction; strong heart contractions lead to increased cardiac output and better heart function. However, this drug has a very narrow therapeutic window, so slight increases in blood glycoside levels may cause the drug toxicity to manifest itself in severe and irreversible side effects, which may be fatal. Additionally, glycosides are very sensitive to blood potassium levels, and low potassium levels enhance the drug's side effects. Several humans secrete glycoside-like peptides, which may interfere and give inaccurate readings for glycoside; therefore, isolation of glycoside before detection. In this embodiment of the ISCCCISCS system of the invention, real-time or near-time ISCCCISCS with a biosensor for glycoside and potassium is placed in the patient's mouth, and alerts are sent to a healthcare provider and/or patient if either one or both blood glycoside and/or potassium levels reach near toxicity levels. The alerts may lead to a reduction in digitalis and or potassium doses. This example of an embodiment of the ISCCCISCS of the invention may shorten the hospitalization of post-surgery patients who are on digitalis, and thus, significantly reduce the cost of their care. In this embodiment of the inventions, any two or more drug interactions may be determined, and the ISCCCISCS system may communicate with different nano-particles in the drugs.

In another embodiment of this invention, equine performance could be measured in real time to enhance performance. During equine sports such as racing, jumping, dressage, and others, electrolyte imbalance, hydration or dehydration levels, respiration, and other biological parameters can be measured in real time to enhance equine performance. During equine racing in hot climate places such as Saudi Arabia, dehydration and electrolyte imbalance could easily set in; therefore, knowing and understanding the equine hydration levels during training is important to ensure that the horses are feed enough water and electrolytes before the beginning of an event for best performance. In fact, racehorses could suffer a collapse and even die as a result of heat stress. Heat stress or heat exhaustion results from electrolyte imbalance and protracted fluid loss during exhaustive exercise among racehorses. The ISCCCISCS installed in either a horse-bit or a cheek or lip clip or patch could help owners and jockey know hydration and electrolyte levels of their horse for enhanced performance and injury prevention.

In yet another embodiment of this invention, the ISCCCISCS of the invention may be used in conjunction with other smart wearables, attachables and insertables anywhere on the body of the same or another individual to form a network of smart wearables or other medical devices used by a collective group such as a basketball, polo team horses, or other sports team. A single or a set of wearable or other medical devices can be placed on the team member/s that send alerts and communications, through the ISCCCISCS in real-time or near-time, to the coach and/or the team's healthcare provider, to ascertain the medical and/or diagnostic condition of each team member for the best sports outcome. The data collected may be used to determine the health status of each and/or to compare with another team member or collectively as a team. For example, a player might be very important to the successful outcome of the game, but he is injured; based on his blood electrolyte levels as determined by the ISCCCISCS his performance can be predicted and the information used to help the coach to make strategic decisions. In another example, the coach compares dehydration statistics between two or more players on the team to decide their performance.

Still, in yet another example of this embodiment, the coach compares the collective statistics of the team as a whole under varying conditions such as weather, geographic location, team acclimation, etc. The ISCCCISCS of the invention, in this scenario, in certain embodiments, also communicates with environmental sensors on inanimate objects to determine performance characteristics of the athletes. In this scenario, sensors for humidity, fatigue-related inflammation, cortisol-related stress, muscle tone, muscle break-down, muscle function, performance, lactose, inertia for gait, accelerometer, and gyroscope, speed, electrolytes, and stamina are all used simultaneously. Measurable data may also be generated on groups or sub-groups of an opposing team. The ISCCCISCS can provide for adjustment of an individual's performance based on, e.g., wearable data analysis.

2. Manual Control Diagnosis Mode.

In addition to the above, the ISCCCISCS user, caretaker, healthcare provider, and others can manually activate one or more biosensor data-points in order to visualize and analyze information inputs from the host animal or human.

In one example of this embodiment of the invention, a biosensor is unable to determine the exact sub-type of cancer cells present in saliva or oral blood supply. The user who could have a higher possibility of cancer due to family history and the user manually turns on the ISCCCISCS. Biosensors for all types of tumor, which represent at most 0.01% of the saliva, are installed on a retainer in the oral cavity. The ISCCCISCS concentrates saliva which further is detected by the biosensor installed to several types of tumor cells based on cell-surface biomarkers, and only 10% of these tumor cells are malignant and cancerous. Therefore, saliva might contain at most 0.001% or less of malignant cells needed to diagnose cancer. At least, 100 milliliters or 0.1 liters of saliva might be needed to collect ten microliters of the malignant cells for analysis in the laboratory. However, these cells do not survive in dilute condition for too long outside of the body; so when outside of a human body, these cells need to be stored properly at subzero temperatures. These cells do survive at high concentrations and when proteases are removed from the saliva. Due to these conditions, it is difficult to diagnose these cancers accurately until malignant cell concentrations in the saliva increases and protease concentration are reduced so that these cells can survive storage after concentration and isolation in ISCCCISC module. However, when more such cells are present in the saliva, the disease could have progressed and could lead to poor prognosis for the patient since by then the disease might have advanced and be difficult to treat. According to this aspect of the invention; the biosensor is allowed to bind to the tumor cells for several hours, whereby a sufficient preset amount becomes concentrated to properly, reliably, and timely diagnose cancer. When a sufficient preset amount binds or absorbs to the biosensor, an alert is sent to remove and send the biosensor or biosensor data acquired for further analysis. The concentrated and isolated cells may be detected by ISCCCISCS, ISCCCIRS, or sent to a laboratory. This situation can be used according to the invention wherein it is substituted by other infectious diseases or other diagnostic molecules that are initially present at sub-diagnosable amounts in infants, children, adults, or animals.

3. Programmed Automated Diagnosis Mode.

In addition to the aforementioned modes, the user of the described ISCCCISCS can automatically activate one or more biosensor data-points in order to visualize and analyze information inputs from a host animal or human. In this embodiment, the ISCCCISCS is set to any schedule (second, minute, hour, day, week, month, etc.) to activate or monitor the medical health of any species or patient.

In this embodiment of the invention, specific symptoms, such as elevated glucose and hemoglobin A1C that predict diabetes may be monitored regularly. Also, alterations in blood concentrations of sodium, potassium, or urea are predictors of a variety of diseases such as Cushing disease, Cystic Fibrosis, kidney diseases. In one embodiment, humans or dogs with diabetes and kidney disease may be on ISCCCISC for blood glucose, Hemoglobin A1C, sodium, and potassium. Daily monitoring during early morning before food ingestion may be programmed because fasting blood glucose levels are the best predictor of diabetes. For a human, normal glucose levels are between 70 and 100 mg/dL, and for the dog, normal glucose is 70-150 mg/dL, so levels above 100 mg/dL for humans and above 150 mg/dL for dogs could send an alert to the healthcare provider. Hemoglobin A1C levels in the body reflect an increase in blood sugar for two to three months, and thus, these should be measured approximately once every one or two months. Hemoglobin A1C levels above 4% to 5.9% may be set to cause the described ISCCCISCS to send an alert to the healthcare provider. Normal sodium levels for humans is between 135 and 145 mEq/L and normal blood potassium levels range between 3.5 to 5.2 mmol/L. When the sodium and potassium levels are higher than 145 mEq/L and 5.2 mmol/L, an alert would be sent to the healthcare provider who can intervene to correct the imbalance in sodium and potassium levels.

4. Geographic Analysis Mode.

In addition to the above, in a further embodiment, a geographical ISCCCISCS dissemination and locator mode are also utilized in order to, for example, monitor and analyze communicable diseases localized in a specific geographical area, a herd of cattle, a herd of sheep, or other animals on a protected geographical range. Local, regional, domestic, or international monitoring and notification is optionally transmitted through the described ISCCCISCS. Training and performance of athletes and racehorses are impacted by altitude, racing conditions, the jockey, training, etc.

Certain diseases can be linked to a local diet, environment, cultural factors, disasters, etc., and the described ISCCCISCS may be applied in these situations. For example, the nuclear spill, such as the one due to an earthquake, in the Kashiwazaki-Kariwa Nuclear Power Plant in Japan, caused humans and animals to be exposed to high levels of radiation. Exposure to high levels of radiation causes a variety of cancers. Early detection of cancer enhances the prospects for a good outcome of most cancer treatment and salivary biomarkers for some cancers appear before any other diagnostic test. In one embodiment of this invention, a panel of various cancer biosensors could be placed in the ISCCCISC for several cancer biomarkers in the oral cavity of the exposed people, and the ISCCCISCS alerts healthcare providers about the development of new signs of cancer in these individuals.

5. Specific Disease or Situational Alerting Mode.

In addition to the aforementioned modes, the ISCCCISCS of the invention is a source of disease-specific or situation specific modes of operation. Local, regional, domestic, or international monitoring and notification is optionally transmitted through the described ISCCCISCS.

Nina has a family history of several diseases, and her doctor sends her a packet with ISCCCISCS to isolate biomarkers for all several diseases that she is in her family history and detect the diseases at early onset. This panel of ISCCCISC sent to her disease specific to her. ISCCCISC could detect blood chemicals such as sodium, potassium, calcium, blood glucose, lipid profile, hemoglobin, hemoglobin A1C, blood creatinine, C-reactive protein, cortisol, family related cancers-biomarkers, and common infections causing microbes. She uses the ISCCCISC as instructed; such as, blood glucose should be used during early morning fasting time, but the cortisol should preferably be used at AM and PM hours for over a few days. Some of the biomarkers would be detected via ISCCCISC, ISCCCIRS could detect others, and some biomarkers would be sent to a laboratory to further analysis. Which sensors would be contained within the device and which sensors would be reader by provided reader, and finally which salivary components would be sent to a laboratory for further analysis would depend upon the concentration of the component in both the saliva and salivary concentrate and sensitivity of the sensor. Components with higher concentration and sensor sensitivity would be contained within the device; whereas, components present at sub-detection levels in the salivary concentrate would be sent to a laboratory for further analysis, and perhaps process, for proper detections. Based on sensor sensitive and other factors that would be determined during development and manufacturing. All values obtained are sent to her physician's office, and her physician may discuss her health status with her during her next visit.

6. Activation and Wake-Up by Another Biosensor, Device, and/or Remote Mode.

The ISCCCISCS described herein includes biosensors located elsewhere within and/or on an animal or human. The ISCCCISCS also interfaces with various other devices located on or near an animal or human, and may securely communicate with a plurality of remote devices that monitor the health and/or well-being of one or more animals or humans. Other diagnostic devices, drug-dispensing devices, other devices, and/or other biosensors are optionally used to activate or wake up the ISCCCISCS S of the invention. The ISCCCISCS may also is optionally remotely woken up by a caretaker, healthcare-provider, and/or others. In yet another embodiment of the invention, the described ISCCCISCS triggers activation and wake-up. These triggers maybe self-induced to control and activate one or more ISCCCISC within the oral cavity in conjunction with remote activation through one or more smart devices. Local, regional, domestic, or international monitoring and notification may be transmitted through the ISCCCISCS of the invention. Further, many examples of embodiments of this invention are possible with several different ISCCCISC, ISCCCIRS, and devices for both humans and animals without departing from the scope of the invention. Also, the ISCCCISCS of the invention includes storing secure data captured by the oral cavity when no connectivity is available. The ISCCCISCS and ISCCCIRS of the invention both stores and/or stream data and/or packet as programmed. Additionally, if ISCCCISCS and ISCCCIRS-captured data is securely stored, it is optionally streamed when connectivity becomes available.

An example of a diagnostic device that may be used in this system, a cardiac monitor is employed to detect abnormalities. When it does so, it sends a signal to activate the described ISCCCISCS to begin monitoring and collecting data about the patient's electrolyte imbalances, blood glucose, and/or prescribed drug blood concentrations. The ISCCCISCS and ISCCCIRS also sent an alert to the healthcare provider who may remotely activate additional biosensors within the ISCCCISCS.

7. Communication Through WiFi and Other Wireless Modes.

In this mode, WiFi, GPS, GPRS, or other secure communications occur between the described ISCCCISCS, ISCCCIRS, and sensors located elsewhere within and/or on an animal or human. The ISCCCISCS and ISCCCIRS also securely communicate through WiFi, GPS, GPRS, or other methods with other devices located on and/or near an animal or human. These communication modes are subject to a plurality of remote devices that monitor the health and/or well-being of one or more animals or humans. Local, regional, domestic, or international monitoring and notification may be transmitted through the described ISCCCISCS. When these communications are lost, the data is stored locally on the device until the communication is restored and the data is streamed to a central secure server.

James is an older adult who lives alone. His nurse visits him weekly to monitor his vitals and other medical symptoms to ensure his well-being. In this example, James suffers from several diseases. James provides the ISCCCISCS kit contains several disease-specific biosensors included in his dentures to diagnose problems with his overall health and disease status. He is provider either with a smart necklace or a smartwatch that he is supposed to wear at all times. These smart accessories contain a built-in WiFi and/or GPS, GPRS, microphone, and speakers. The ISCCCISCS as described could be remotely turned on by the nurse or physician to measure specific biosensors when the blood, stool, or urine collected weekly by the nurse suggests abnormal levels of a biologic. This further saliva testing could ensure that James is doing well and his medication dosing could be changed before next visit by the nurse. In this example, certain circumstances could save James live and at the same time reduce healthcare cost.

8. Other Combinations Using the ISCCCISCS System.

In addition to the above, any and all combinations could be exemplified for a multitude of ISCCCISCS purposes and uses. A real- or near-time, programmed, etc. pertain to multiple ISCCCISCS applications.

In one embodiment of this invention, mare pregnancy could be determined non-invasively and earlier than currently available methods. Best current method to determine mare pregnancy is a blood test serum gonadotrophin (PMSG) level, which is measured between 40 and 100 days after breeding. The hormone is released about days 36 to 38 of pregnancy, and the PMSG is first detectable in the mare's blood after about day 40 of fertilization. However, by about day 130, PMSG production has ceased, and it is no longer detectable in blood. Therefore, this test is not recommended after 120 days of pregnancy. One major problem with the PMSG blood test is that it can give a “false positive” results if the mare has been pregnant but loses the fetus, which is common among mare. One fastest way to detect pregnancy is to measure hormones that rise and fall using during mare's menstrual cycle. One such hormone is P4, and pregnancy can be determined in about 10-15 days of fertilization. P4 levels increase significantly within the first 12 hours of ovulation and could reach about 2 ng/ml by 48 hours post-ovulation. In non-pregnant mares, P4 levels rapidly decline after day 14 of the cycle. In mare's saliva P4 levels increase concurrently with serum P4 levels after ovulation, peaked by day 8 and subsequently declined after day 14. Salivary P4 levels rise and fall in conjunction with serum levels during the estrous cycle in cycling mares and also during early gestation in pregnant mares. ISCCCISCS containing P4 collecting, concentrating, detecting modules could be added to equine smart-bit. The ISCCCISCS could also be removed and either send for further analysis or read by a reader. The advantage of this method is pregnancy detection is within about 10-15 days of fertilization, a better understanding of menstrual cycle for a particular mare, and this non-invasive and convenient method. This understanding of an individual mare's menstrual cycle could help in determining best time periods for fertilization of a mare.

In this embodiment, the ISCCCISC can be used to determine fertility, ovulation, and pregnancy. The ISCCCISCS system can be set to a combination of automated, real-time or near-time and manual modes. It is understood by anyone familiar with the art that in females, basal body temperature and several hormones associated with menstrual cycles and pregnancy change during the menstrual cycle. For example, during the ovulation phase of the menstrual cycle, follicle stimulation hormone (FSH) and Leutenizing Hormone (LH) from the pituitary glands are elevated. During pregnancy, the pregnancy announcing hormone, human chorionic gonadotropin (hCG), appears elevated in the blood, but during labor several hormones, including oxytocin, adrenaline, and endorphins become elevated. ISCCCISCS for various aforementioned hormones may help in the diagnosis of the onset of ovulation, pregnancy, labor and some diseases related to fertility by use of real- or near-time, manual, and programmed automated alerts.

According to embodiments of the invention, different tier, degree, and intensity levels of alerts are possible with each one of the alert modes. Basal measurements for any diagnostic parameter can be set at the lowest level of alert for each mode. Some alerts can be established as repetitive alerts, and bi-directionally transmit out alerts at pre-set interval until alert-receipt is acknowledged by the receiver.

ISCCCISC and ISCCCIRS-related biomarkers and applications: these devices optionally are used for a variety of diseases including, but not limited to, oral and systemic infectious diseases, cancers, drug abuse, metabolic diseases, malnutrition, obesity, cardiovascular diseases, atherosclerotic inflammation, sleep disorders, stress, fatigue, stroke, and still-to-be-discovered disease-signature and disease-linked biomarkers. While all of these and other applications, with a variety of oral biosensors, would yield significant information, the information without the communication and alert systems would not be nearly as useful to human and animal patients, caretakers, healthcare providers, and or others. This communication could not be possible without in-situ collection and concentration system that would make sub-optimum salivary components detectable. The communication and alerting system, along with the oral biosensors, is incorporated into the invention ISCCCISC and ISCCCIRS. In addition to the oral biosensors, the ISCCCISC and ISCCCIRS of the invention includes other biosensors that interface with the ISCCCISC and ISCCCIRS, and are located elsewhere, within, and/or on an animal or human. The ISCCCISC and ISCCCIRS are capable of interfacing with a variety of other devices and nano-particles that are located in or on or near the animal or human, and can securely communicate with a plurality of remote devices that monitor the health and/or well-being of one or more animals or humans. The ISCCCISC and ISCCCIRS of the invention also include secure data system software with visualization modes and presentation system software. The latter feature is available to utilize a variety of platforms including, but not limited to, charts, graphs, histograms, and/or bar graphs. Caretakers, healthcare providers, etc. can visualize data including, but not limited to, correlations and comparisons between different episodes and historical data for patients within the same species, breeds, and/or disease categories; or within different species, disease categories, and/or situations. For example, data from laboratory animals can be correlated and extrapolated to other animals or humans suffering from the same or similar diseases. Such kinds of scenarios are possible because the secure data system contains data captured and/or collected from various alerts. The secure data system also inputs data into the system to set alerts, which can be analyzed and visualized by the ISCCCISC and ISCCCIRS software. Some of this data will be viewable and available to everyone, so will be public, yet some data will be viewable and available only to relevant individuals and healthcare providers, and hence will be private. The secure data system also inputs data into the system to set alerts, which can be analyzed and visualized by the ISCCCISC and ISCCCIRS software according to this aspect of the invention.

It is to be understood that the above description is intended to be illustrative, and not restrictive. For example, the above-described embodiments (and/or aspects thereof) may be used in combination with each other. Additionally, many modifications may be made to adapt a particular situation, method, system device, development, need, manufacturing requirements, regulation, or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claims.

We claim:

1. A system comprising a device configured to be securely inserted in an oral cavity of an animal or human comprising a receptacle for oral component collection, concentration, and storage.

2. The system of claim 1 wherein, the receptacle being configured to serve one or more functions within the animal or human's oral cavity without being swallowed, the device further comprising one or more smart sensors contained within or upon the receptacle, and at least one interface with a network configured to utilize the information obtained from the one or more sensors or from one or more platforms.

3. The system of claim 1 wherein, the receptacle being configured to isolate one or more oral components.

4. The system of claim 1 wherein, the system comprises one or more network units configured to carry out a functionality selected from the group consisting of signaling bi-directional transmissions to a secure server through one or more of Wi-Fi, MiFi, Wi-Fi Direct, Bluetooth, ZigBee, Internet of Things, GPS, and NFC, temporarily storing information in the smart device, bi-directionally transmitting alerts to pre-selected devices or pre-selected personnel.

5. The system of claim 1 wherein, the system comprises one or more units for local digital data storage independent of wireless storage, the stored data collected through any digital storage device, connector, or mechanism.

6. The device of claim 1 wherein, the means by which the receptacle is securely attached within the oral cavity of an animal or human comprises one or more of being fixedly inserted, imbedded, fitted, fixed, implanted, fastened, joined, associated, coupled, linked, banded, united, mounted, combined, glued, adhered, cemented, or firmly connected by mouthparts or hands or accessories thereto.

7. The device of claim 1 wherein, the receptacle is insertable by micro- and nano-clips, frames, brackets, sealants, dental composites, bonds, adhesives, adhesive strips, cements, wires, bands, glues, embedment, injection, printing, tattooing, or any combination thereof.

8. The system of claim 1 wherein, the receptacle is connected to a trans-dermal blood extractions module for blood component extraction, collection, concentration, and storage.

9. The device of claim 1 wherein, the receptacle being configured to contain at least one or more of permeable membrane, semi-permeable membrane, non-permeable, synthetic membranes, biological membranes, coated membranes, uncoated membranes, color changing membranes, microfiltration membrane, ultra-filtration membranes, nano-filtration membranes, osmotic membranes, reverse osmotic membranes, ionic or charged membranes, neutral membranes, size-exclusion membranes, affinity membranes, adsorption membranes, extracting membranes, binding membranes, hydrophilic membranes, hydrophobic membranes, electrostatic attraction and repulsing membranes, polar membranes, non-polar membranes, lipophilic membranes, lipophobic membranes, coated membranes, resistant membranes, capillary action membranes, suction generating membranes, micro-pumps, absorbent materials, volume changing materials, pressure generating membranes, surface tension generating membranes, energy releasing membranes, energy absorbing membranes, coated nano-beads, coated micro-beads, gel, and reactive membranes.

10. The system of claim 1 wherein at least one of the one or more functions of the device is selected from the group consisting of replacing missing and/or repairing teeth or parts of teeth and the receptacle is permanent or temporary caps, implants, night guards, partial guards, crowns, jacket crown, jacket, dental plate, denture, plate, removable space maintainers, bridges, partial or full dentures, dental implants, veneers, whitening traces, fillings, fixed prostheses, braces, dental wires, partial or full retainers, prostheses, artificial teeth, prosthodontics, inlays, onlays, sealants, dental composites, bonds, temporary materials, permanent materials, removable materials, materials used in dentistry, materials used in tongue piercing, adhered onlays or inlays, moldable materials, materials embedded, cemented or adhered to a palate, inside of cheeks, lips, tongue, sublingual cavity, gums, or teeth, teeth aligning, fixing, or malpositioned teeth or jaws, retainers, braces, space maintainers, headgear, palatal expanders, fixed prostheses, braces, dental wires, partial retainers or full retainers, veneers, whitening and cleansing strips, and professional, amateur, or lay-person cleansing tools and equipment, and whitening or cleansing traces; pacifying for infants, children, adults, elderly, and animals, pacifier-like device, mandibular advancement devices (MAD), tongue retaining devices (TRD), continuous positive airway pressure (CPAP), combination CPAP/dental sleep device therapy, a night guard or partial guard, a full or partial mouth guard, tongue piercing, bendable and/or flexible ISCCCISCS patch, stretch stripes, adhesive stripes, ISCCCISCS patches or tattoos with electronic insert and/or cartridge and/or a lab-on-a-chip, a tongue protector, an attachment to a tooth, inner-lips, or inner-cheeks, inner lip clip, inner cheek clip, an insert in a gum, gum-cover, a smart gauge, smart dipstick, smart rod, smart stick, horse-bit; a thermometer, an attachment to a tooth, inner-lips, or inner-cheeks, inner lip clip, inner cheek clip, an insert in a gum, gum-cover, a ISCCCISCS gauge, ISCCCISCS dipstick, ISCCCISCS rod, ISCCCISCS stick, biteplate, bendable or flexible ISCCCISCS device unit, electronic insert, stretch stripes, adhesive stripes, anesthetic machine for continuous-flow, vaporizer, oxygen mask, nasal oxygen mask, Guedel airways, oral Suction catheter, ventilator or mechanical breathing machine, respiratory maintenance circuit, Laryngoscope, Intubation Tube, Laryngeal mask airway, endotracheal tube, endoscopes, eschmann stylet or gum elastic bougie, respirator, mucus sucker, chew toy for babies and animals, and flexible, inflexible, temporary, or permanent ISCCCISCS.

11. The device of claim 1 wherein, the receptacle comprises of one or more sensors of blood pressure, core body temperature, heart rate, optimum breathing, levels of a predetermined biologic, chemical, medication, drug metabolites, the touch sensors, contact temperature, non-contact temperature, infrared, pressure sensors, volume-sensing, gas or liquid pressure, absolute pressure, differential pressure, vacuum pressure, gauge pressure, conductive rubber pressure, lead zirconate titanate pressure, polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel, a light sensor, an active amplifier, light dependent resistors, optical filters, fluorescence, phosphorescence, sound, resonant, humidity, changes in humidity via measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

12. The device of claim 1 wherein the one or more sensors measures cells, cancerous cells, biomarkers for an oral or systemic infectious disease, biomarkers for drug abuse, biomarkers for a metabolic disease, biomarkers for malnutrition, biomarkers for obesity, biomarkers for a cardiovascular disease, biomarkers for atherosclerotic, biomarkers for infection, biomarkers for auto-immune and other immune diseases, biomarkers for stroke, biomarkers for AIDs, biomarkers for multiple sclerosis, biomarkers for periodontal diseases, biomarkers for brain-function disorders, dementia, memory loss, depression, mental disease, Alzheimer's disease, mentally-challenged disorders, nervous system disorders, tracking or wandering, and other psychology and neurological disorders, biomarkers for bleeding, head and neck injuries, biomarkers for Sjogen's syndrome, biomarkers for oxidative stress, biomarkers for allergies, biomarkers for cancer, biomarkers for skeletal and muscle diseases, biomarkers for genetic diseases, biomarkers for renal diseases, biomarkers for osteoporosis, biomarkers for fatigue, biomarkers for stress, biomarkers for sleep deprivation or sleep apnea, biomarkers for fertility, pregnancy, ovulation, and reproductive system disorders, biomarkers for cystic fibrosis, biomarkers for respiratory or pulmonary diseases, biomarkers for diabetes and ketoacidosis, biomarkers for inflammation, biomarkers for age-related diseases, biomarkers for dehydration, biomarkers for halitosis, biomarkers for alcohol consumption, alcoholism or drug consumption or drug addiction, biomarkers for hypoxia, smoking-related diseases, toxins, or pollutants, biomarkers for poor-gait, biomarkers for Crohn's disease, biomarkers for Cushing disease, biomarkers of stress, biomarkers for dental caries, biomarkers for blood and circulatory disorders, biomarkers for ear, nose, and throat diseases, biomarkers for taste, Ageusia, Hypogeusia, or Dysgeusia, biomarkers for bad-breath related diseases biomarkers for chewing or mastication, biomarkers for digestive disorders, biomarkers for hepatic diseases, spleen, gall-bladder and pancreatic diseases, biomarkers for urinary system disorders, biomarkers for integumentary system diseases, biomarkers for endocrine, lymphatic, and excretory diseases, sensors which measure a cell surface component or a cellular marker or component, sensors which measure a pathogen or a microbe, sensors which measure administered foreign materials, medications, diagnostic molecules, drugs, biologically sensitive, derived, bio-mimics, or bioengineered molecules, sensors which measure an ingested molecule or its metabolite, including wherein ingested molecule is a pathogen, a microbial, an ingested toxin, an ingested allergen, an ingested food constituent, including a nutrient, a micronutrient, a fat molecule, a carbohydrate molecule, a sugar molecule, a protein molecule, or an amino acid, sensors which measure ingested medications, ingested foreign material, ingested drugs, an ingested diagnostic molecule, an ingested biologically sensitive molecule, an ingested nano-particle, an ingested derived molecule, a bio-mimic, or an ingested bioengineered molecule, lymphatic, and excretory diseases.

13. The system of claim 1 wherein the system comprises at least one auxiliary smart sensor receptacle not configured to be inserted in an oral cavity of an animal or human.

14. The device of claim 1 wherein, a part or entire part of the oral device is removable for further processing and analysis by a “reader,” the reader further comprising one or more smart sensors, and at least one interface with a network configured to utilize the information obtained from the one or more sensors or from one or more platforms.

15. The system of claim 14 wherein the system comprises one or more network units configured to carry out a functionality selected from the group consisting of signaling bi-directional transmissions to a secure server through one or more of Wi-Fi, MiFi, Wi-Fi Direct, Bluetooth, ZigBee, Internet of Things, GPS, and NFC, temporarily storing information in the smart device, bi-directionally transmitting alerts to pre-selected devices or pre-selected personnel.

16. The system of claim 14 wherein, the system is configured to be connected to a smart device comprises one or more network units configured to carry out a functionality selected from the group consisting of signaling bi-directional transmissions to a secure server through one or more of Wi-Fi, MiFi, Wi-Fi Direct, Bluetooth, ZigBee, Internet of Things, GPS, and NFC, temporarily storing information in the smart device, bi-directionally transmitting alerts to pre-selected devices or pre-selected personnel.

17. The system of claim 14 wherein, the system is configured to be connected to a smart digital phone, which further comprises the light source as excitation energy source, photodiode, Light Emitting Diode, camera as a light measuring device, optical photo, density, colorimeter, video and audio recorders to measure or record changes in biological activities, the touch sensors or pressure sensors to measure changes in volume, pressure, and temperature.

18. The device of claim 14 wherein, the receptacle comprises of one or more sensors of blood pressure, core body temperature, heart rate, optimum breathing, levels of a predetermined biologic, chemical, medication, drug metabolites, the touch sensors, contact temperature, non-contact temperature, infrared, pressure sensors, volume-sensing, gas or liquid pressure, absolute pressure, differential pressure, vacuum pressure, gauge pressure, conductive rubber pressure, lead zirconate titanate pressure, polyvinylidene fluoride pressure, PVDF-TrFE pressure, FETs pressure, metallic capacitive sensing elements pressure, resistance, tactile, elasto resistive sensors, conductivity, color, luminance, movement, optical, photo sensors, photo detectors, pixel, a light sensor, an active amplifier, light dependent resistors, optical filters, fluorescence, phosphorescence, sound, resonant, humidity, changes in humidity via measurements of mass, a mechanical, electrical changes as moisture is absorbed, could be used to measure humidity, or changes in temperature of condensation, changes in electrical capacitance, resistance, dielectric constant, dew point hygrometer, thermal conductivity temperature, or thermal conductivity, magnetic, electromagnetic, position, ionization, pH measurements, electrodes, fundamental electrical measurements, piezo-electric, piezo-resistive, potentiometric, orientation, video, 2 or 3D images, density, mass, MEMS, Lab-On-Chip to Micro Total Analysis, biosensors, chemo-sensors, biologic-sensor, a biologically-relevant-sensor, blood chemicals, blood electrolytes, pH, blood oxygen level, respired gases, gases, optimum breathing, oral air-flow, gyroscopic measurement, accelerometer measurement 1D, accelerometer measurement 2D, accelerometer measurement 3D, kinematics, ionic conductivity, photos, videos, images, electrical waves, sound waves, spectrophotometry, electromagnetic spectrum, gamma waves, X-ray wave, ultraviolet waves, visible waves, infrared waves, terahertz waves, microwaves, radio waves, magnetic waves, ultrasonic waves, magnetic resonance, magnetic field, electro- or magnetic-encephalography, functional magnetic resonance imaging, optical topography, global positioning or tracking, and radiation wave activity.

19. The system of claim 14 wherein, the system comprises one or more units for local digital data storage independent of wireless storage, the stored data collected through any digital storage device, connector, or mechanism.

20. The system of claim 14 wherein, the system comprises at least one auxiliary smart sensor receptacle not configured to be inserted in an oral cavity of an animal or human.
